(19) United States
(12) Patent Application Publication
(10) Pub. No.: US 2005/0245902 A1
(43) Pub. Date: Nov. 3, 2005

(54) Substance delivery device

(76) Inventors: Brian Cornish, Hamilton (NZ); Andrew Philip Oakley, Hamilton (NZ)

Correspondence Address: ABELMAN, FRAYNE & SCHWAB, 666 THIRD AVENUE, 10TH FLOOR, NEW YORK, NY, 10017, US

(21) Appl. No.: 10/504,237

(22) PCT Filed: Feb. 10, 2003
(86) PCT No.: PCT/NZ03/00019

§ 371 Date: Jul. 15, 2005

(30) Foreign Application Priority Data
Feb. 8, 2002 (NZ) 517094

(51) Int. Cl. A61K 9/22

(52) U.S. Cl. 604/890.1

(57) ABSTRACT

A substance delivery device capable of insertion into a body cavity of an animal, with the delivery device including a body capable of housing a delivery apparatus capable of actively being controlled to autonomously deliver at least one substance into a body cavity of an animal, with the delivery apparatus including dedicated pressure systems to deliver the at least one substance from independent reservoirs via at least one associated outlet, with the at least one substance ranging in form from substantially fluid to substantially solid, with the device also including a programmable control device capable of initiating and regulating delivery of the at least one substance in accordance with a preferred delivery regime, and with the body further including a tail portion adapted to receive retention apparatus external to the animal and capable of effecting retention of the substance delivery device within the body cavity of an animal.

### TECHNICAL FIELD

This invention relates to improvements in and relating to a substance delivery device for autonomously delivering in situ one or more substances, in either or both solid and fluid forms, from separate reservoirs via substantially dedicated outlets, to an animal. The substance delivery device includes delivery apparatus and programmable control means for effecting controlled delivery of each substance from the device, in accordance with a preferred delivery regime. Accordingly, the substance(s) are delivered to the animal in predetermined quantities, for predetermined dose duration, at predetermined times to effect desired concentrations of the substance in the animal as required to effect a desired outcome. The substance delivery device also includes means for attachment of retention apparatus to maintain the device in situ during the period over which the device is required to deliver the substance(s) from the substance delivery device.

Reference throughout this specification shall be made to the substance delivery device as being used for the introduction of substances within a naturally occurring body cavity, for example such as for intra-vaginal and intra-rumenal applications. Whilst the device has particular application in domesticated and/or farmed mammals, such as cows, horses, pigs, sheep, goats, dogs and so forth, the device may also have application in a far greater range of farmed or breeding animals (such as deer, llamas, etc) and may include zoo animals and endangered species.

It should be appreciated however, that the principles of the present invention can also have far wider applications than this and can be used in any appropriate situation where autonomous delivery and some degree of control of delivery of sometimes multiple substances, in a variety of forms (through the continuum from solid to liquid to gaseous substance(s)) are required.

In particular, the delivery device is adapted to receive complementary retention apparatus that will enable the substance delivery device to be most commonly used in situations where the device, capable of insertion into a passage or body cavity of an animal, is required to be retained in the passage or body cavity for varying lengths of time depending on the delivery regime of the delivery device being implemented. Such passages or body cavities are cavities generally associated with the reproductive or digestive systems of an animal, but include the ear, vagina, uterus, stomach, rumen, rectal cavity and so forth. Such devices are inserted into an animal's body cavity where control, synchrony, regulation and so forth of a biological function, or status of an animal is required. Examples include control of parasites, improved nutrition, synchronized reproduction, regulated growth, or delivery of a medicament and so forth and where this control is effected through chemical, mineral, vitamin and/or hormonal intervention, and may include applications tailored for humans.

However, the present invention could have further applications outside this field. Accordingly, these delivery devices may not necessarily be within the body of animals, but may be used for other purposes, such as horticultural, industrial, domestic, and so forth.

### BACKGROUND ART

Commonly, substance delivery devices are inserted into a living animal for dispensing substances to the animal for various reasons. Typically, these include the administration of a chemical, hormone, nutritional supplement and so forth depending on the desired outcome.

To this end a number of subcutaneous implanted substance delivery devices including programmable control means and delivery apparatus for dispensing doses of a medicament as continuous flow and/or pulsatile doses, are available in the prior art. These devices have been developed for the introduction of medicaments, such as insulin for diabetes, for example. As such these devices are small and suited to the delivery of one substance (typically in liquid form) into surrounding tissue, or the blood stream. They are however, not suited to delivery of multiple substances, nor to delivery of substances in a variety of forms (along a continuum of gases, liquids, suspensions, pastes, powders, tablet/capsules). They also rely for their retention on being inserted into a surgically created pocket below the skin and as such are not suited for use in varyingly sized, naturally occurring body cavities; where the issue of retention is more problematic.

A range of other substance delivery devices have been developed for retention within a passage or body cavity of an animal. These devices include intra-rumenal devices and intra-vaginal devices. Some of these devices may rely on the use of helical springs, expanding coils, lobes, elongate arm-like projections of variable geometry hinged or attached to a device and so forth for retaining the device in the cavity for the required period.

Some retention systems may also rely on muscle tension in the body cavity to assist with maintaining the devices in place These devices are typically used as intra-vaginal contraceptive devices for animals. In the anterior vagina smooth muscles bands are arranged longitudinally and latitudinally around the vagina. Pressure from these muscles is often relied on to retain devices in situ in the vagina. However, the pressure applied by the muscles is not always applied in a manner to effect total retention of the device. Therefore, retention apparatus associated with the device is often adapted to improve device retention. For example, the length of any lobes, arms, wings and so forth when fully elongated may be wider than the diameter of the cavity. Therefore, there is tension applied to the anterior walls accordingly. In some instances, more than one kilo of force can be applied to the anterior vaginal wall and it is strictly a forced effect against the anterior vagina that contributes to retention of the device. In yet other devices, the anchoring mechanism places sufficient pressure on the vaginal wall to alter the normal cross-sectional configuration of the vaginal tract. This in turn, puts tension on the animal which may be noticeable when the prior art device is inserted into the cow, in that there is often an immediate physical response by the cow. That the cow feels the presence of the product may be evidenced by the cow hunching up, due to the tension felt on the anterior vaginal wall.

In yet further systems, the protrusions from the device may actually be required to embed into the vaginal mucosa in order to effect, or improve retention.

In devices including extendable retention systems which typically rely on the application of some degree of pressure against an area of the wall of a body cavity of an animal, or are embedded therein to effect the retention, removal of the device may be difficult, or may cause some damage to the walls of the cavity or the associated passage. However, others may rely on collapsible systems for removal of the device. Yet others may rely on being biodegradable over time, such that after releasing an active ingredient (due to the animal's body fluids acting to leach out the active ingredients often impregnated into the body, or part thereof, of the device) into the animal the surface area decreases and the devices is able to be expelled from the animal.

Others may rely on weighted systems, particularly for rumenal application. These retention systems and/or delivery devices per se are typically bulky and are designed to prevent expulsion of the device through peristaltic, muscular pressure applied to the device, or through regurgitation.

Bulkiness of the retention system can however be a further source of discomfort to the animal. Some may be too bulky to pass back through passages leading into or out of the body cavity when the functional utility of the device has been exhausted. Therefore, for weighted intra-rumenal devices, the devices are never intended to be removed, but rather remain inside the animal for the animal's lifetime. As can be imagined the number of spent devices may be significant if a number of repeated treatments are effected.

Where bulky devices are used for intra-vaginal applications, the surface area of such devices may impede the flow of body secretions, particularly the flQw of mucus in the vagina of the animal. This in turn may impact on the transfer of the actives (released from the device) through the walls of the body cavity and, by extension, the bioavailability of the actives in the animal's blood as required to effect the desired outcome.

A tendency for mucous generation is a frequent occurrence using intra-vaginal devices, due to the presence of a foreign body which actively works against the vaginal mucosa. Where such devices secrete a hormone, particularly progesterone, excess mucous generation may be a natural reaction by the animal to this presence. Excessive generation of mucous can actually absorb progesterone and can affect the release profile of progesterone through the vaginal mucosa. Further, if extreme pressure is applied to the anterior vagina this causes irritation and can also result in extreme generation of mucous.

The vaginal secretions themselves are self-cleaning until normal peristaltic flow from the vaginal cavity is obstructed and this tends to lead to infection. Enclosed loops, or wherever there is an area where mucous can be trapped, actually causes interference in terms of the normal functioning of the animal. This is because the mucous forms a film or a build up of semi-glutenous mucous holding onto the device around that structure, stopping the normal peristaltic flow. Prevention of normal peristaltic mucous flow can eventually lead to infection internally.

Other problems with such devices may include, prevention of penile insertion during attempted intermission by the male animal.

Devices directed to retention in a rumen of a beast typically have arms are that are rigid but resiliently and hingedly attached at a juncture on body. Such arms may be made from nylon which is rigid to make it inflexible but still provide some ability to allow the arms to collapse during insertion. Their purpose is not to contact surfaces of the rumen in order for the device to be retained, but rather they are designed to open up so as to prevent regurgitation. Any sort of degree of flexibility along their length would be unsuitable as this increases the possibility that the shape is altered and the device is be able to be regurgitated. Such devices are typically designed to be present in the rumen long term

However, problems associated with this device include creation of crystallization of the nylon arms at the juncture with the body. This often results in inherent brittle weakness at the point, often further resulting in arms being broken off and the device subsequently being regurgitated.

With some devices used for intra-vaginal application, it has been noted that the device is not able to remain appropriately placed and may be capable of completely spinning around inside the animal with the tail end of it being up towards the cervix. Other prior art devices include anterior wings that face forward and provide tension to the vaginal wall. In fact, they face into the cervical area and when splaying out, physically distend the vaginal cavity near the cervix which can cause problems of irritation of the cervix. Such devices are not suitable for the purposes of delivering hormones for breeding or reproduction because there is such a high degree of interference around the cervix and, as most treated animals are artificially inseminated, it would require such devices to be removed at the time of breeding.

Accordingly, it would be desirable if the substance delivery device was easily inserted and removed, yet was reliably retained for required periods, did not impede flow of body secretions nor adversely affect normal functioning by its mere physical presence, nor detrimentally impacted on the wall of the cavity.

Similarly, a range of delivery apparata for delivering substances are well known, and have broad application. Many incorporate pumps and are used to dispense common substances. Those used inside a living animal are used to dispense substances such as chemicals, nutritional supplements or drugs. A number of delivery apparata are known that introduce substances such as hormones intra-vaginally to cows to attempt to promote the onset of oestrus. This enables the farmer to artificially inseminate the cows at a time when they are most fertile.

Others contain useful substances impregnated into or coated onto various parts of the device and rely on administration of the active into the animal gradually over a period of time by the action of body fluids. Accordingly, such apparatus typically relies on the natural processes of diffusion, dissolution, or osmosis to dispense the substance. As such these types of delivery devices operate on a passive delivery regime. To effect release of different actives into the animal's body at different times some delivery apparata have a number of layers, perhaps having different thickness, containing both the active substances, which may be released sequentially.

However, when effectively controlling oestrus in animals for example, the complexity of the process naturally requires any artificial intervention introduce different hormones in appropriate quantities and concentration into the animal's body at particular times. Passive delivery of substances into the animal cannot be controlled as it is totally dependent upon the environment that it is in. The rate of introduction of the substances may be dependent on a number of factors including temperature, mucus concentration, salt concentration, kinetic action and so forth of the body fluids. These factors are variable from animal to animal which leads to variable timing and concentration of the substances being introduced into the body. Passive release of multiple hormones may not produce reliable or consistent results. Hence, such systems tend to restrict themselves to delivery of a single active material, or if more than one, the second material is introduced manually into the cavity or via intramuscular injection.

When considering, conventional pumps for delivery of multiple substances, the problems encountered include issues of size. Conventional pumps are often large and complicated. They contain moving parts, complex valve systems, are usually difficult to operate with any degree of accuracy, and are often not suitable for insertion into an animal's body. Further, conventional pumps often require regular maintenance.

For those systems adapted to be small enough for use in the body, such as delivery devices implanted subcutaneously (whether operating on positive pressure principles or not), they tend to have one pumping/delivery system that is controllable to deliver only one substance. Further, the substance is typically in one form—usually a liquid. Any variations to the delivery regime may depend on increased concentration or increased quantity of active which is effected by a bolus or pulsatile dose delivered intermittently to a continuous flow, but at the same time and through the same delivery outlet on the device. There are no facilities for delivering different actives, in different forms, from different outlets, at different times, through the operation of differently configured delivery apparatus and in accordance with a delivery regime established to effect the desired outcome.

It would therefore be desirable if there could be provided a delivery device which was substantially small and simple, contained no complex parts, was maintenance free, and which could be used in animal's bodies or other such environments.

It would also be desirable if there could be provided a device which included delivery apparatus capable of being accurately operated to autonomously deliver multiple substances in different forms if required, into an animal in precise concentrations, in precise quantities and with precise timing, for precise durations and where such apparatus could operate independently of the environment, or the apparatus could release substances into the environment only when the environment was ideal.

In addition, it would be desirable if there could be effected some control over the delivery apparatus after the device is placed in the animal, and/or if there was some way of determining what was happening within the animal with respect to the operation of the device and associated delivery apparatus.

It is an object of the present invention to at least address the above problems or at least to provide the public with a useful choice.

Further objects and advantages of the present invention will now be discussed by way of example only.

### DISCLOSURE OF INVENTION

According to one aspect of the present invention there is provided a substance delivery device capable of insertion into a body cavity of an animal, said delivery device including a body, the body capable of housing delivery apparatus capable of actively being controlled to autonomously deliver at least one substance into the animal's body cavity, said delivery apparatus including dedicated pressure systems to actively expel the substance(s) from independent reservoirs via associated outlet(s), said substance(s) ranging in form from substantially fluid to substantially solid, the device also including programmable control means capable of initiating and regulating delivery of the substance(s) in accordance with a preferred delivery regime, the body further including a tail portion adapted to receive retention apparatus external to the animal and capable of effecting retention of the substance delivery device within the body cavity of an animal.

This invention relates to improvements in and relating to a substance delivery device for autonomously delivering in situ one or more substances, in either or both solid and fluid forms, from separate reservoirs via substantially dedicated outlets, to an animal. The substance delivery device includes delivery apparatus and programmable control means for effecting controlled delivery of each substance from the device, in accordance with a preferred delivery regime. Accordingly, the substance(s) are delivered to the animal in predetermined quantities, for predetermined dose duration, at predetermined times to effect desired concentrations of the substance in the animal as required to effect a desired outcome. The substance delivery device also includes means for attachment of retention apparatus to maintain the device in situ during the period over which the device is required to deliver the substance(s) from the substance delivery device.

The term substance used in this specification shall mean a substance in any form along a continuum from substantially solid to substantially fluid. Accordingly substances may be housed and/or delivered in substantially solid forms (including tablets, capsules or micronised powders), in a substantially liquid form (including gels, pastes, suspensions, solutions), or in gaseous forms. Further, the substances may be delivered by release of solid tablets/capsules in situ, as a spray, as droplets, as a continuous flow and so forth.

With reference to one preferred embodiment of the invention, the substance delivery device will be described with reference to an intra-vaginal delivery device, developed to deliver required hormones in required doses at required times into the anterior vagina of an animal for the purpose of synchronising oestrus. The animals with which this device may be used include a range of farmed and/or breeding mammals, zoo animals and endangered species. For the purpose of describing the device and its application, reference will be made to an intra-vaginal embodiment used with cattle for the purpose of synchronising oestrus for effecting controlled breeding programmes. Whilst an intra vaginal device is exampled, the invention may be adapted for use as substance delivery devices for intra-rumenal, intra rectal use and so forth.

The anterior vagina in a cow is a roughly tubular-shaped cavity. The dimensions vary between animals depending on the animal per se, whether it has previously calved, how old the animal is, how many calves she has had and also how long after calving. For example, if the animal is, 30-40 days post-calving then the cavity is going to be looser than in an animal that has had a long duration of time between calving and the time of treatment, or an animal that has not calved at all. The anterior vagina is made up of smooth muscle forming longitudinal and latitudinal bands. The muscle bands encircle the complete cavity and are peristaltic by nature. The anterior vagina also has a mucosal lining. The mucosal lining maintains vaginal moisture through mucous secretions. Peristaltic waves tend to push the vaginal secretions back towards the outer vagina. Such peristaltic waves determine the need for a retention system typically employed with intra-vaginal devices. Thereby, in effecting retention of a substance delivery device within the anterior vagina there is reliance on the smooth muscle characteristics yet at the same time there is a need to be able to cope with the peristaltic waves that are generated during the period the device is inserted in the animal.

The remaining description of the substance delivery device will relate to one preferred embodiment used as an intra-vaginal device for the purpose of synchronising oestrus in cattle. Whilst the delivery device in the described embodiment is an intra-vaginal device, it should be appreciated the device may be adapted for use in other specific areas of the animal's body—for example, as an intra-rumenal, intra-rectal device, and so forth.

Further, although the ensuing description relates to the delivery device for use in synchronising oestrus in cattle and to the delivery of formulations (including preferred hormones and preferred carried solutions) to effect that synchrony, it should be appreciated this invention may have application in the delivery of a range of formulations (via appropriately adapted devices) into other areas of the body to control or synchronise other body functions/cycles.

This exampled embodiment of the invention operates as a controlled breeding device used to deliver formulations to synchronise the oestrous cycle in cattle for fixed time blanket insemination in either cycling or non-cycling cows. This is achieved by the accurate delivery of a complex hormone regime through a preferred system involving control (preferably electronic programmable control) of a unique pumping system.

The preferred device ensures accurate delivery of 4 different hormonal formulations to the animal at precisely the required dose and at the exact time during an appropriate “x”-day treatment period, such as a 9-day, or an 11-day treatment period, for example. Further discussion relating to the formulations and the design concept relating to the delivery apparatus and control means of the device to effect delivery of the formulations, is discussed later in this specification.

For ease of reference the substance delivery device capable of insertion into a body cavity or passage of an animal shall now be referred to from time to time simply as the device, although it should be appreciated this term is not intended to be seen as limiting.

In preferred embodiments of the present invention the substance delivery device includes a body. The body is a substantially hollow receptacle. The body is configured to include a chamber capable of housing the chassis of the device with which the delivery apparatus and associated controlling apparatus (including a power source as required) is associated. The body is further adapted to receive attachment means for appropriate retention apparatus used with the device.

The function of the body is to protect the internal components and formulations from animal fluids and also to protect the device during transportation and handling prior to insertion. The body has a secondary function in that it also provides the means of attaching the retention system to the device.

The body of one preferred embodiment includes two separate, but inter-connectable portions. For ease of reference the two portions of the body shall be referred to as the front portion and the rear portion, however, use of these terms is not intended to be limiting. The front portion is typically the most anterior, or leading portion when the device is inserted into the animal. The rear portion is located at the trailing end of the device.

The front portion of the body of this preferred embodiment is substantially elongate and cylindrical in shape, having a substantially uniform cross-sectional profile. The rear portion is a substantially larger-diameter ovoid cylinder, being substantially shorter than the front portion. However, it should be appreciated that in different embodiments the structural configuration of the two portions may vary. In addition, the body may include more than two portions. The possible variations will be dictated by a number of factors including the intended cavity and the physiology of the animal into which the device is being inserted, the number of substance formulations and form of the substances to be delivered and hence the number and structural configuration of reservoirs required to house the formulations, the delivery apparatus (both type and quantity) required to deliver the formulations, the number and location of the outlets for the substances, the control means (including the power source used) for effecting the delivery regime of the formulations, which in turn is dependent on the process for which the device is being used and the length of time the device is to be retained in the animal.

In the front body portion, a section of the wall of the body is substantially thinner and includes an indented portion. This indented portion preferably coincides with the location of particular internal mechanisms of the device, and more specifically the on/off switch of one embodiment. Accordingly, the thinner body wall in this portion has been created as part of the design feature to allow an operator to be able to activate the on/off switch of the control means of device externally by depressing the switch through the thinner body wall, thereby effecting manual activation of the device before it is inserted into the animal. As can be appreciated, in other embodiments where activation of the device is effected by remote means, such as electronic signaling and so forth, the requirement for the on/off switch and/or the indented portion of the body may be optional. In other embodiments also, the body may include thinner walled portions elsewhere as required to effect whatever function is desired, whether it is manual activation of a switch, whether it is to improve visibility of a particular component, such as an LED display, an indicator light, and so forth.

The front part of the body is also preferably adapted to allow for orifices/apertures or the like that form the outlet(s) from which the substances in the reservoir(s) are delivered. For example, in the embodiment being described, the front body portion includes an aperture where the outlet pin for a progesterone reservoir is located, even though the reservoir for this substance may not necessarily be encased by the front body portion. For example, in the embodiment being described, the progesterone reservoir is located towards the rear of the fully assembled device, yet the outlet pin is located flush against the outside surface of the front body portion.

The front portion may also be capable of receiving a nose cap portion substantially at the leading distal end. The nose cap portion is preferably configured to include outlet orifices. The outlet orifices are associated with conduits/reservoirs dedicated to containing separate substances. The cap portion preferably includes hinged, removable sealing means for delivery of the multiple substances from the reservoirs when required in accordance with the preferred delivery regime. When sealed, the sealing means provide a substantially fluid tight seal to each of the substance reservoirs. The substances are expelled from the reservoirs/conduits by the controlled operation of pressure devices associated with each reservoir. The delivery process causes release of the removable sealing means, allowing the contents of the substance reservoirs to be delivered into the animal. The reservoirs/conduits are associated with an internal chassis portion of the device (to be described later) and may be enclosed within either or both the front and rear portion(s) of the body.

The front portion of the body is also further adapted to preferably locate onto the internal chassis of the device. The front portion so locates via a slot cut into the body, which matches to a corresponding complementary configured ridge located on the chassis, although other locating means may be employed with the invention to effect the purpose.

The rear body portion or rear cap, as it may also be called, essentially covers at least one substance reservoir. In the embodiment being described, the reservoir preferably houses a liquid progesterone formulation. However, in other embodiments, one or more reservoirs may be located in this region, for housing one or more substances in substantially liquid (including pastes or gels), or substantially solid (including micronised powder or capsules/tablets) or gaseous form.

The rear body portion also preferably features a breather hole in the area relating to the progesterone reservoir. Subsequent development from earlier designs identified that a breather hole was preferably required to equalise the air pressure within the device as the progesterone reservoir was emptied during dosing. This is now a permanent feature of the preferred product design where collapsible reservoirs are used.

The rear portion, or rear cap, in this described embodiment also preferably includes a recess on the horizontal access at the rear of the cap for insertion of a tail rod. The tail rod is force-fitted to the rear body cap during assembly. The tail rod provides a secure anchor point for attachment of the retention system to the rest of the device.

In this preferred embodiment, the inside of the rear body cap may also be strengthened with a series of plastic ridges or such like to provide more rigidity to the rear portion. Improved strength in this region is useful to support both large fluid filled reservoir(s) and/or the tail structure. Both of these may result in forces being applied to the rear portion, which in turn could have the propensity to collapse if not strengthened.

The rear body cap features a locking system to the front body via a number of recesses moulded into the rear body portion. These recesses are capable of receiving and interlocking with complementary configured barbs on the front body portion. However, any suitable configuration for effecting interconnection of the body portions may be adapted for use with this invention. The connection area between the two body components is also preferably strengthened to allow the two components to fit together without distorting and running the risk of coming apart while in use. Preferably, the rear cap and the front portion are interconnected during the final assembly stages of the device when the two halves are married together and joined as a press-fit. Once joined together, the two parts are preferably inseparable other than via destruction of the device. It is important that the device does not fall apart whilst inside an animal.

The outside of both body portions preferably has a very smooth finish. The body is preferably made of thermoplastics material. The plastic used is preferably polypropylene, although a softer grade of material is preferably selected. The smooth, soft plastic body and the cylindrical shape is so configured to improve the ease with which the substance delivery device is able to be inserted into and withdrawn from a passage or body cavity of an animal, with minimal difficulty, without effecting damage and/or discomfort to the animal.

Preferably, the body portions lock over the chassis to protect the chassis and its internal components from animal fluids.

The chassis of the device may be described as the skeleton of the product. From the chassis, all of the other components are attached. However, as can be appreciated, the shape of the chassis will evolve over a number of iterations in accordance with the structural features of the embodiment of the device with which it is used. As discussed previously, the device body will vary in configuration due to a number of factors. The same factors will similarly impact on the structural configuration of the chassis.

The chassis of one preferred embodiment, apart from simply being required to house and hold the components in one place, features reservoirs for up to or more than three front reservoirs, a mechanism for containing the printed circuit board and for housing valve and delivery pressure systems, where required, for both front reservoirs and rear reservoirs. For example, valve and delivery systems for additional liquid substance reservoirs, such as a liquid progesterone reservoir located at the rear distal end of the device, are located relative to the chassis.

In earlier embodiments, one possible chassis design was used in conjunction with a one piece external body which was slid forward over the chassis to lock against some external wings that slotted into some body slot cavities.

However, in other embodiments, where the body includes two or more portions, the chassis unit slots into the front portion of the body via a locating lug. Further, in such an embodiment, the body itself is not actually physically clamped, fixed or retained to the chassis but instead to the chassis locating lug slides. The chassis is further retained in place via a spring/resilient delivery system used with the currently described embodiment, which forces the chassis against the front body section and via O-rings.

The O-rings further operate to keep moisture from the animal's vagina away from the internal working mechanism, including the electronics, of the device. The number of O-rings may range between two and three O-rings located around the chassis which press against the body. For example, in the currently described embodiment, two O-rings are featured. However, more (or less) O-rings may be employed as required, depending on the where the device is used.

The importance of the O-rings to effect a fluid-tight seal for the internal electronics is particularly relevant in embodiments including venting holes to allow atmospheric pressure to equilibrate during progesterone dosing. It is important to recognise that such venting holes in the body allow animal fluids into the device (particularly into the progesterone reservoir area) during the equilibrating process. Accordingly, the chassis, needs to be moisture-proofed to prevent any ingress of moisture into critical areas of the device

In the preferred embodiment described herein, the chassis is approximately 116 mm in length and has a maximum radius of 22.6 mm. However, the physical dimensions of the chassis may vary between iterations of the device, or where different devices are manufactured for different uses or with different animals.

In one embodiment of the present invention, the chassis is manufactured from polypropylene plastic.

The intra-vaginal device is preferably made of pharmacologically safe materials, for use in animals, that also do not react with the formulation compounds and solutions being administered by the device. Thermoplastic materials are preferably used. Such materials included in and on the device, that are exposed to contact with the treated animal are listed as follows:

| | |
|---|---|
| Device body | polypropylene copolymer |
| Tail | polyurethane elastomer |
| Chassis | polypropylene |
| Nose cap | polypropylene |

All plastic materials exposed to the animal are in compliance with FDA regulation 21CFR 177.2600 and are suitable for food contact grade applications.

The tail of the device is the portion of the device that contributes both in part to insertion of the device into an animal, as well as to retention of the device within the animal's cavity (where the cavity is the vagina). The tail extends from the rear of the body of the device to the exterior of the animal's vulval lips. In effect, the tail comprises an internal connecting portion for the retention apparatus for the device.

According to another aspect of the present invention there is provided a substance delivery device substantially as described above wherein the tail includes a substantially internal shaft portion and an external retention apparatus retaining portion (located at the outer distal end of the tail).

In preferred embodiments of the present invention the shaft has a substantially limited cross-sectional dimension. For ease of reference the substantially limited cross-sectional feature of the shaft of the tail rod shall now be referred to as thin, although the use of this term is not intended to be limiting. A thin shaft facilitates retention capability of the device without impeding the flow of body secretions, or causing discomfort or injury to the animal. Unimpeded flow of body secretions is necessary for the normal biological functioning of an animal's reproductive system, and so forth.

In some embodiments of the present invention however, the shaft may be thicker, or have varying cross-sectional dimensions, depending on the strength of the peristaltic waves in the body cavities into which the device is inserted and depending on the types of body secretions present in the different body cavities of an animal.

In preferred embodiments of the present invention the shaft is substantially elongate to enable the tail to be attached to the delivery device, yet be long enough to extend the length of the vaginal canal and be available for attachment to the retention apparatus, whilst also being of sufficient length to minimize the likelihood of the delivery device being dislodged or expelled from the anterior vagina. The retention apparatus retaining portion is substantially angled with respect to the shaft portion and is adapted to receive a portion of the retention apparatus.

Accordingly, in one embodiment, the tail protrudes from the rear of the body (once fitted) by a distance of approximately 126 mm. The tail rod is substantially circular in a cross-section and is approximately 14 mm in diameter along its length. The distal portion is angled at right-angles to the shaft and extends for approximately 12 mm prior to the commencement of the barbs where the external retention apparatus is attached. Whilst these dimensions and configuration is suited for use with the delivery device to be inserted into the vagina of a cattle beast, it can be appreciated that other embodiments used for other species may vary considerably. Apart from variations to the length, cross-sectional shape and thickness, the tail rod may be configured other than a substantially straight rod. For example, portions along part or the whole length may be spiraled, undulating and so forth.

According to another aspect of the present invention there is provided a substance delivery device substantially as described above wherein the tail is capable of being used to facilitate insertion or withdrawal of said substance delivery device into a body cavity of an animal.

According to another aspect of the present invention there is provided a method of inserting into and withdrawing from a body cavity of an animal, a substance delivery device substantially as described above.

In one preferred embodiment described herein, the tail serves as the applicator for the new device by enabling the person inserting the device to hold the tail at the time of insertion of the device and to use it to guide and deliver the device to the correct location within the animal.

The retention apparatus retaining portion (located at the outer distal end) of the tail is substantially angled at a right angle. Whilst this configuration is designed to effect easy attachment of the retention apparatus, it also serves to determine the depth of insertion as this right angle is placed against the vulval lips of the animal. However, it should be appreciated that the outer distal end portion of the tail may be otherwise configured yet still effect the same functions.

In preferred embodiments of the present invention the device is capable of being removed from the passage or body cavity of an animal. Once the functional utility of the device has been exhausted, the device may be withdrawn by pulling on the tail portion, assisted by the pressure exerted by the walls of the passage or the opening to the body cavity. The pressure enables the device to be withdrawn with minimum discomfort to the animal.

According to another aspect of the present invention there is provided a substance delivery device substantially as described above wherein the tail rod for the retaining apparatus occupies a plane substantially aligned to the central axis of the body of the substance delivery device.

The tail rod is preferably rigidly fixed into the body of the device such that when connected to the retention apparatus the orientation of the tail is pre-determined and causes the device to have the same orientation within the animal in all instances.

In one preferred embodiment, the tail is located into the rear body of the device. In other embodiments, the tail may be locatable with respect to the chassis. The tail is preferably secured to the body by press-fitting the tail rod into an aperture in the rear portion of the body. The tail is preferably secured via a number of plastic barbs, which positively engage with the softer material of the body. However, any other suitable attachment configurations may be employed, or adapted for use with the invention. Once the tail is fitted to the body, the fit is preferably permanent and the tail becomes irretrievable without destruction. Although, in other embodiments where different attachment configurations are used, the tail may be detachable and/or re-usable.

In other embodiments the tail rod may be completely moulded onto the body of a device. In yet other embodiments the shaft of the tail rod may be extendible telescopically. Telescopic extension of the shaft may enable it to be adapted to suit a range of sizes of body cavities or passages into which the delivery device may be inserted.

The exterior surface of the tail is preferably smooth so as not to cause any abrasion or trauma with the target animal. The smooth surface of the tail also ensures that any animal excrement, mucous or body fluid does not bond or bind to the tail once inserted and while in situ.

In preferred embodiments of the present invention the shaft is substantially circular. Having a substantially circular shaft avoids the possibility of sharp edges causing irritation of the interior walls of the animal's body cavity. In addition, substantially circular, thin shafts are inherently stronger than thin, flat shafts for example. Further, a rounded surface is less likely to impede flows of body secretions to the same extent that a flat surface might.

However, in other embodiments of the present invention, the shaft may be flat, V-shaped, U-shaped, hexagonal, and so forth, depending on the internal conditions of the body cavity into which the device is inserted, and the corresponding shaft strength required.

In preferred embodiments of the present invention the shaft is substantially straight. Although, in other embodiments it may be curved; undulating, helical and so forth, to meet the particular needs and/or physiology associated with different body cavities of different animals.

The material from which the tail rod is manufactured is an important consideration, as it is required to be subjected to stress without the shape becoming distorted. In addition, this material needs to be durable, have smooth surfaces after moulding to minimise risks of contamination to the animal, needs to be capable of being moulded, capable of being sterilised for hygienic reasons, be lightweight, chemically resistant, withstand wet environments, and be economical.

Materials suitable for use as the tail rod were assessed through a series of tests which identified the tail rod needed to be made of a very rigid material. A series of different plastics (such as Hytrel™, nylon, acetyl, polyethylene, polypropylene and various grades of glass-filled nylon containing various contents of glass—from 5% to 40%) were tested. In tests it was identified that low levels of glass (below 25%) included in nylon/glass rods provided too much flexibility for the tail rod. High levels of glass (percentages greater than 33%) resulted in difficulties with the injection-moulding process and often resulted in a less-than-smooth surface finish to the moulding. Nevertheless, a range of glass/nylon combinations from between 10% and 40% could be suitable in a variety of situations.

The tail in preferred embodiments is manufactured from Du Pont Zytel™ 331, which is a 33% glass, reinforced with nylon 66%. It is a very rigid, non-flexible material. Any flexibility may negatively impact on the ability of the tail to be used in the insertion of the device, or may jeopardise the retention of the device in the animal for the length of time required to effect delivery of the substances into the animal. However, any suitable material may be used for the tail provided the material enables the tail to function as required.

Whilst one preferred material from which the tail is manufactured is glass-filled nylon (because it provides rigidity), in the event of it being broken this material provides a very sharp, rigid spike from the rear of the device, which has the potential to perforate the vaginal wall and, theoretically, cause peritonitis and kill the animal. Therefore, the tail rod may be made from any suitably rigid material to effect the required performance of the tail rod, whilst at the same time balancing the rigidity with other concerns, such as the potential for injury.

According to another aspect of the present invention there is provided a substance delivery device substantially as described above wherein the angled distal portion of the shaft is adapted to receive at least a portion of the retention apparatus.

In preferred embodiments, the tail is an integral part of the retention system. The tail is substantially the internal portion whilst the external portion of the retention apparatus is attached to the delivery device via the tail of the delivery device.

The rear distal end of the tail rod of one embodiment includes a series of protrusions, such as barbs. The barbs provide a suitable surface against which a portion of the external retention apparatus may interact via a friction fit. Barbs were selected as the ideal attachment means for fitting the external retention apparatus because they were deemed to be compatible with the material from which the external retention apparatus is preferably made. For example, the external retention apparatus is preferably made from rubber and the elastic nature of the rubber, the ease of moulding a cavity in the end of the rubber to fit over the rod and the hard nature of the rod material against the soft pliant rubber, ensures a non-removable fiL The combination of the rod-to-rubber fit is particularly successful because no other component is required to ensure the fit, it is a cost-effective option and, importantly, it keeps the diameter of the rubber and tail rod to the minimum diameter which suits the animal application.

The tail rod has a number of advantages. The most notable feature of the tail rod and retention system is that no internal pressure is generated against the vaginal wall while the device is in situ. The tail rod provides a positive fit of the elastic retention system to the device, ensures that the device remains in the correct orientation while in situ, and ensures minimal trauma to the animal during insertion and removal. Further, most if not all other existing intra-vaginal systems require an applicator for the insertion of the various devices. No applicator is required for the present invention

According to another aspect of the present invention there is provided a method for retaining a substance delivery device substantially as described above within a body cavity of an animal into which the substance delivery device is inserted, via use of retention apparatus.

The purpose of the actual retention system is to retain the device in the correct location in the body cavity for the required duration. A secondary consideration for the retention system is to minimise the amount of trauma as indicated by physical discomfort or any negative physical change potentially caused by the retention system. It is also a requirement of the retention system that the product can be removed from the animal easily at the appropriate time and also without causing any negative impact.

According to another aspect of the present invention there is provided a substance delivery device substantially as described above wherein the retention apparatus is attachable at the rear distal end of the substance delivery device when the device is inserted into an animal's vagina.

In preferred embodiments of the present invention the retention apparatus is attachable at the rear distal end of the device. Accordingly, the attachment of the retention apparatus to the device occurs externally of the animal. This in turn requires an external point of attachment of the retention apparatus on the animal.

This enables the retention apparatus' to be attached in appropriate positions on the outside of the body of the animal as required to enable the device to be more reliably retained within the animal's body cavity. The positioning of the retention device is largely dependent on the animal's behavioural characteristics. For example, naturally passive animals, such as cows may allow the retention apparatus to be attached to the back of the animal. However, naturally inquisitive animals, such as pigs may easily pull on and remove retention apparatus attached in such locations. Therefore, it may be necessary to ensure the retention apparatus surrounds only the vicinity of the vulval area, relying on the animal's reluctance to allow other animals to touch that area to minimize the likelihood of another animal pulling off, or chewing the retention apparatus.

In one preferred embodiment, the retention apparatus comprises a patch and strap arrangement. The patch is attached to the back of the animal. The strap is retained under sufficient tension, and along with the configuration of the delivery device is adapted to reduce the likelihood of the delivery device simply being expulsed from the animal via muscular peristalsis, or being accidentally pulled from the vagina. Attachment of the patch to the animal's back is an important consideration. If the retention rubber strap is too tight at the time of insertion and gluing of the patch to the back of the animal then the upright portion of the tail has the potential to cause severe trauma to the vulval commissure.

In other embodiments, the retention apparatus may include a cap, diaphragm or similar means that applies suction or adhesion around the vulval region and retains the device in situ. However, any other suitably configured external retention apparatus may be adapted for use with the invention.

In preferred embodiments the external retention apparatus includes a substantially elongate rubber strap with a circular patch at one distal end and means of attachment of the strap to the angled portion of the tail rod at the opposite distal end. The rubber strap is preferably injection-moulded and has elastic properties enabling it to be force-fitted over the distal end of the tail rod where the protruding barbs are located. Further development of the strap may include using an alternative to the rubber strap but still using a material that displays elastic/resilient properties.

The patch is preferably approximately 55-60 mm in diameter and is designed to be adhered to the back of the cow. One side of the tail strap patch is glued to the animal. This side has a roughened surface to accentuate the bond between the strap and the cow when the adhesive is applied. Rather than applying adhesive on site prior to attachment for the patch, the patch may be pretreated with adhesive and may simply require removal of a backing tape to expose the adhesive surface prior to application. Another alternative to the rubber patch is a nylon mesh or similar type mesh to be glued to the back of the cow rather than a solid rubber object. This is because it has been identified that solid rubber may, in fact, cause excessive sweating underneath the patch when adhered to the animal and, in some cases may cause failure of the adhesive. However, any suitable material may be used for the patch as required.

Retention results using this preferred retention system have demonstrated a number of advantages. Generation of mucous is substantially minimised (even when alcoholic solutions are used with this retention system), animal retention has been found to be extremely positive averaging 98-99%. The amount of trauma or animal irritation typically due to the presence of the device in the anterior vagina may now be reduced due to the body of the device being smooth and cylindrical, rather than including protruding internally located retention apparatus common in the prior art. The only incidence of trauma occurring is where the rubber strap is fitted too tightly, causing the tail rod to abrade against the vulval commissure.

In preferred embodiments of the present invention the retention apparatus is attached to the device capable of being inserted into the vagina of an animal. Although, in other embodiments of the present invention the retention apparatus may also be attached to a device capable of being inserted into other body cavities or passages being accessible from outside the animal.

In preferred embodiments of the present invention the retention apparatus is manufactured separately from, but attachable to the device. Having retention apparatus which is a separate portion, enables the retention apparatus to be attached to a device at a later stage of construction; enables the retention apparatus to be made of different materials to the body of the device; may enable a particular form of retention apparatus to be used on a number of different devices; or enables one device to be fitted with any one of a range of retention apparatus depending on the body cavity into which the device is to be inserted.

However, in other embodiments of the present invention the retention apparatus may be an integral part of the tail and/or body of a device to which the retention apparatus is attached. Having the retention apparatus as an integral part of a device obviates problems of having to attach the retention apparatus as a separate portion at a later stage of assembly. Further, it enables the same material to be used for both the body of the device and the retention apparatus. Therefore, where the retention apparatus is moulded as an integral part of the tail and/or body of a device, the expense and time associated with construction of the retention apparatus and device may be reduced.

According to another aspect of the present invention there is provided a substance delivery device substantially as described above wherein the retention apparatus is substantially flexible. As previously mentioned, the retention apparatus may be made of materials, such as rubber. In addition, the retention apparatus is preferably made from a material which is capable of being moulded, capable of being sterilised for hygienic reasons, is lightweight, chemically resistant, can withstand wet environments, and is economical.

In preferred embodiments flexibility of the retention apparatus contributes to its ability to remain in the appropriate location without applying undue pressure on the animal, or without effecting, contributing, or enabling removal/loss of the delivery device from the passage or body cavity, whether through it being dislodged by the animal's internal peristaltic movements, or by other external means.

According to another aspect of the present invention there is provided a substance delivery device substantially as described above wherein the retention apparatus is applied externally of the animal.

Having the retention apparatus attached externally of the animal obviates problems associated with the retention apparatus increasing or impeding the flow of body secretions, such as intravaginal mucus, or causing discomfort to the animal or causing damage to the interior walls of the cavity (the vagina) during insertion, retention or withdrawal of the device.

In determining the most efficacious design of the delivery device it was identified that the device, apart from being retained in the animal for the treatment period, needed to be able to house and deliver the required formulations to the animal in a controlled way.

The embodiments described herein are referenced with regards to the use of the device for synchronizing oestrus in cattle. The desire to control oestrus onset has been promoted by changes to farming practices which focus on improved breeding regimes. For example, in a group of dairy cattle, at the time of breeding there are about 25-30% of them that may be deemed infertile. In other words, they are physiologically capable of reproducing but due to some environmental effect (it could be stress, nutrition, and so forth) they are producing large volumes of milk and also therefore producing hormones that suppress fertility. They are effectively not able to conceive because they are not having a normal ovulation process. By definition, this means they are infertile. The balance of the cattle in the population, whether 70-75%, may be typically cycling at different times. One of the major challenges in modem agriculture is to be able to effectively identify when those cows are in the ideal stage to conceive and therefore be inseminated (since breeding of dairy cattle is mostly achieved by artificial insemination).

Therefore, when looking at the treatment regime for the delivery device, it was identified that a drug regime was needed that was capable of treating both fertile and non-fertile cattle without having to discern the difference between the groups of cattle. This is because any diagnosis of the fertility status is open to human interpretation and therefore failure. It has been determined that to effect synchrony of oestrus in cattle (in the example used to describe one embodiment of the invention herein) requires delivery of more than one hormone, in suitable concentrations and quantities, to be released at specific times and for specific durations, and in appropriate forms with carrier compounds as appropriate to enable the hormones to be effectively transported across the vaginal mucosa to effect blood plasma concentrations required to effect the desired response.

Therefore, it was further identified that the drug regime would need to be controllably delivered by appropriate delivery apparatus within a suitably configured delivery device.

It was determined that four hormone formulations would be required to synchronise/control/regulate oestrus. These formulations need to be delivered at strategic times to be able to effectively treat the animals. The drug regime therefore developed involves delivery of two formulations of estradiol, one of prostaglandin and a progesterone formulation over a duration of time. The formulations needed to be delivered in a manner that was effective, not only in terms of delivery but also in terms of being able to be absorbed by the animal upon delivery in the desired manner. For example, steroids are non-water soluble and for the active ingredient to be absorbed readily by the vaginal mucosa the steroid has got to be presented to the mucosa in a manner that allows it to become readily available in order to effect the desired outcome.

Based on knowledge of the required drug regime and the affect wanted, an appropriate design concept for the delivery device was thereby determined. It was identified that a need existed for a controlled timed delivery of single-unit doses for some hormones. This particularly applied to the hormones previously administered in the prior art by either an intramuscular injection or via a capsule that (in the prior art) was delivered manually. The process was therefore assimilated into an autonomous delivery system by developing the delivery device having delivery apparata that could deliver the required single-unit doses at a specific time. The single unit delivery features of the invention will be described more fully later.

Within the same preferred design concept a need was recognised for continuous dosing of yet another hormone formulation to replicate the naturally occurring situation in the animal. The hormone in question is progesterone. Tests indicated that a hormone solution retained within a collapsible reservoir would meet this objective.

A number of delivery challenges were encountered during development of the delivery apparatus and control means for delivering the progesterone formulation. For example, progesterone has a number of very difficult characteristics.

Firstly, it is poorly water-soluble, so to improve its solubility a solvent or carrier is preferably used with the hormone active (progesterone). The progesterone may either be dissolved in the carrier solution in readiness for delivery, or the carrier solution is stored separately from the hormone active until prior to release of the formulation into the animal, when the carrier solution is introduced to the hormone active. The dissolved final formulation is then either released into the animal, or the mixing occurs at the time of release. The hormone active may be available (for mixing with a carrier solution) in liquid, powdered or tablet form. Although in some embodiments gaseous forms of an active may be appropriately delivered. In one embodiment, the solvent includes an alcohol carrier to solubilise the progesterone. In other embodiments the carrier solution may be, or include a chemical compound such as a suitable cyclodextrin capable of forming inclusion complexes for transferring insoluble, or partially soluble substances across water-soluble membranes.

A second issue to consider in delivering the progesterone formulation is that the total volume being delivered over the entire progesterone treatment period (of typically 8 to 10 days) is relatively small. Thirdly, progesterone has a half life of only 20 minutes. Therefore, it was determined that very, very small quantities of progesterone formulation were required per dose to effect the desired blood serum levels over the progesterone delivery period. This required a delivery system that was very, very precise and could be monitored, measured and allowed accurate calibration/re-calibration to be undertaken.

Accordingly, where the substance delivery device releases an active ingredient such as progesterone, not only is it required that the retention apparatus maintain the device in a position whereby the active ingredient released from the body of the device may be free to mix with appropriate bodily secretions, or be absorbed through the walls of the body cavity into which the device has been inserted, but that the actives be delivered appropriately. This requires having regard to the issues of solubility, the form in which the active substance is released, the means of delivering the active, where the active is delivered, the ability to house the quantity of active required over the total delivery period, and so forth.

In many existing prior art substance delivery devices for use inside bodies or other such environments the substance has been passively delivered by the natural process of osmosis, diffusion, or dissolution. The rate and volume of substance delivery is therefore determined by the natural processes and as such cannot be controlled or varied by artificial means. By comparison, delivery apparatus of the present invention (including pressure devices and/or valve means) provides a means of actively delivering the substance. Further, active delivery of a substance provides a means of controlling the rate and volume of the substance delivered.

As can be appreciated, delivery apparata may take many forms. A number of different delivery mechanisms already exist in the prior art that may be adapted to effect the desired delivery. Piezo pumps are one example. For example, by using two Piezos in tandem a preferred dose may be delivered from the Piezo element and through a restrictor nozzle. Pressure is applied to the fluid formulation via a first Piezo. A small quantity of formulation is then directed to a delivery chamber from the operation of a second Piezo. The formulation can then directed to the outlet of the device.

However, for the purpose of the present invention the delivery apparatus is required to be controlled to actively, yet precisely, deliver often multiple substances.

According to a further aspect of the present invention there is provided delivery apparatus for delivering a substance to an outlet of a substance delivery device substantially as described above wherein the delivery apparatus includes at least one conduit capable of containing the substance, at least one pressure device capable of applying pressure to the conduit, and valve means, the delivery apparatus characterised by application of pressure by the pressure device and activation of the valve means causing the substance within the conduit to move along the conduit to the outlet

The mechanism by which the delivery apparatus actively introduces the substance is via a pressure device, and/or valve means. The term pressure used in this specification shall mean the application of force, and the term pressure device shall include any mechanism used to apply force (such as a pump or spring and/or plunger system). The pressure/force applied to the substance(s) being delivered from the substance delivery device is preferably an expulsive force. For ease of reference the pressure devices may be referred to as pumps. However, it should be appreciated that use of this term is not intended to be limiting.

In terms of the progesterone delivery, one preferred embodiment requires the progesterone formulation to be preferably a liquid formulation. Although a gel, gas, or a solid formulation may be used in other embodiments. The progesterone is preferably dissolved in solution. Preferred solvents such as Marlophen NP3 and/or Propylene glycol P1000 and/or 2-Phenylethanol may be used, although ethanol and water are also suitable solvents. Use of cyclodextrin carriers may further improve the solubility of the progesterone solution.

According to a further aspect of the present invention there is provided a method for delivering a substance to an outlet of a substance delivery device substantially as described above including at least one conduit capable of containing the substance, at least one pressure device capable of applying pressure to the conduit and valve means, characterised by the steps of applying pressure to the conduit and valve means, causing the substance within the conduit to move along the conduit to the outlet.

With a liquid formulation, the delivery apparatus of the device is required to deliver a quantity of active material in formulation according to the preferred delivery regime. The progesterone solution is preferably delivered as a continuous programmed series of pulsatile doses according to command.

According to a further aspect of the present invention there is provided a substance delivery device substantially as described above, including a delivery device for delivering a substance to an outlet including at least one flexible conduit capable of containing the substance, at least one pressure device capable of applying pressure to the flexible conduit, characterised by the application of pressure by the pressure device causing the substance within the conduit to move to the outlet, wherein the pressure device is a pump, a spring and/or plunger system.

According to a further aspect of the present invention there is provided a method for delivering a substance to an outlet of a substance delivery device substantially as described above, via delivery apparatus, said delivery apparatus including at least one flexible conduit capable of containing the substance, at least one pressure device capable of applying pressure to the flexible conduit, characterised by the application of pressure by the pressure device causing the substance within the conduit to move to the outlet, wherein the pressure device is a pump, a spring and/or plunger system.

The pumps are preferably economical pressure devices which have a low power draw and therefore can be readily battery powered.

The term conduit used in this specification shall mean any apparatus capable of conveying a substance and may in some instances be a reservoir. The conduit may be substantially flexible or substantially rigid, depending on the substance conveyed within it, the form the substance takes and the pressure device used to convey the substance within the conduit. Other embodiments may include separate reservoirs also.

The conduit may be divided into two ends. One end, hereinafter referred to as the inlet, is connected to the substance source. The opposite end, hereinafter referred to as the outlet, is connected to the outlet of the delivery apparatus, of the substance delivery device. Where there are separate conduits for multiple substances, each (or some of the) outlet may be dedicated to releasing only one substance from one conduit (or reservoir).

Substantial trial and error occurred before effecting a reliable delivery system to effect delivery of the progesterone solution as a continuous programmed series of pulsatile doses according to command. Among the delivery challenges was the use of a number of mechanical valves and a number of moving seals. These were evaluated with a preferred design concept that was eventually adopted whereby pressure was applied to the back of a collapsible reservoir by a seal and spring. A valve was then opened for a specific period of time to allow some of the contents of the reservoir to fill a small delivery cavity. Pressure from repeated operation of the valve effectively drove the fluid from the device to the outlet.

For ease of reference, the reservoir shall now be referred to as the bellows. However, use of this term is not intended to be limiting.

In preferred embodiments, the bellows (reservoir) is firstly evacuated under vacuum and then the bellows is filled with the appropriate quantity of progesterone formulation. The reservoir is filled so it is devoid of air and is maintained under positive pressure by means of a spring force applied to the back end. Due to the positive pressure, fluid is always presented to the inlet of an associated micro-pump to which the reservoir is attached regardless of the attitude of the device. More specifically, the micropump is operated by means of a magnetic core that is forced to stroke when the electromagnetic coil, within which it is housed, is energised. The core activates a release mechanism that allows a predetermined quantity of solution to enter the micropump. The positive pressure in the reservoir ensures the solution enters the micropump and is expelled from the outlet under pressure. The micropump is very energy efficient to enable small low energy power cells to be used.

In one preferred embodiment, the progesterone bellows is a separate component to the chassis. In other embodiments the progesterone reservoir/bellows may be an integral part of the chassis. It is preferably manufactured from low-density polyethylene plastic, which is blow-moulded, to form a collapsible reservoir. Polyethylene has been selected as a material to use in the bellows because of its compatibility with the formulations used in the embodiment of the device described herein (whether water-soluble progesterone formulations, or certain alcohol-based formulations). However, due to the soft polyethylene material used for the bellows, all testing has been preferably conducted at 39° C. to represent the plastic characteristics at body temperature. At this body temperature, it is notable that the ethylene mouldings become more pliant and this may affect their behavioural characteristics when; the device is in situ. Development of the bellows design was effected through trial and error, with assessments of performance only possible once the bellows was in a moulded form.

The bellows preferably features a number of annular rings. The annular rings enable the bellows to collapse in concertina fashion due to spring pressure directed at the rear end of the bellows and allow the fluid contained within the reservoir to be expelled. The rear end of the bellows reservoir is shaped to provide even collapsing of the bellows. The bellows may be varyingly configured according to the quantity of fluid required to be delivered. Various models have been developed, ranging in capacity from 12 ml to 42 ml.

It is important that the centre of the bellows does not collapse before the annular rings. This is particularly important for bellows that exceed 12-15 ml capacity. With a larger diameter reservoir it can become difficult for the spring to evenly apply pressure to the reservoir. If even spring pressure is not applied then as the bellows collapses the uneven spring pressure causes the bellows to rack to one side of the body. This may cause implosion from the rear end of the bellows, or uneven dosing or output from the device. To minimise racking, the rear end of particularly the larger bellows is preferably strengthened with a cross or supporting structure within the ethylene moulding. Alternatively, in larger diameter bellows arrangements, a seal guide type configuration may effect even transfer the energy from the spring to the bellows unit to minimise the racking opportunity. In such embodiments, the use of seal guides may also require the use of silicone lubricants.

The bellows in one preferred embodiment is open-ended at its anterior end. As the bellows collapses fluid contained therein is directed towards the anterior end of the bellows. A valve is located at the anterior end of the bellows. The bellows is able to couple directly to the valve via a screw-thread, which provides the means of attachment to a valve.

Preferably, the portion of the bellows unit at the valve end of the bellows is tapered to the point where it joins to the valve to ensure the maximum possible amount of progesterone formulation can be delivered to the valve. Failure to have a leading taper to the valve will result in approximately 20-25% of the progesterone formulation remaining trapped within the bellows at the end of the dosing regime.

Some advantages associated with the bellows reservoir are that once the valve arrangement is threaded on to the end of the bellows, it provides a self-contained reservoir for the progesterone formulation. This lends itself to separate filling of the bellows during the assembly process and assists in the commercial manufacture of product. Further, there is a high degree of reliability and dose consistency when the bellows is configured to operate correctly within the device. In addition, previous problems experienced with a moving-seal design have been eliminated and there is good consistency of dose performance between devices. Whilst leakage of progesterone formulation was often experienced with delivery apparatus including moving-seals, little or no leakage has been experienced with any of the bellows designs and a positive fit between the valve and the bellows has been achieved.

The term valve used in this specification shall mean any means, including automatic or other device, used to apply force to, or capable of conveying, a substance from the conduit to the outlet. In some embodiments the valve means may be different from and/or replace one or all of alternate pressure devices. With a metering valve system, such as that described herein, tension apparatus may also preferably be included, such as a spring, to ensure that the valve system is able to reliably seal, when required.

The bellows valve (or progesterone valve for the purpose of this exampled description) comprises a number of fixed and moving components, both metallic and plastic, that are fitted together to form the valve. The valve is responsible for sealing the progesterone reservoir and preventing inadvertent release of the formulation to the outlet as well as allowing the progesterone formulation to be released according to the specification.

The valve features a small hole in the very center, with a sealing face. Against the sealing face, a silicone flap is positioned. A valve ring is applied over the silicone flap to lock it into place. This arrangement is then threaded onto the bellows, which results in the bellows being an integral reservoir.

The valve may operate in one of two ways. The preferred operation is for the spring energy that is transferred against the bellows reservoir, to in turn, cause the progesterone fluid to press against a silicone seal. This keeps the valve closed at all times while the valve is not being operated. A second although less preferred valve design which comprised a small valve (called the mag valve) which included a seal that not only provided a sealing function, but also provided during its operation, the energy to push the progesterone fluid to the outlet.

In one preferred embodiment the valve means is a metering valve system, which operates on a reversed magnetic polarity principle; and includes a moving and a fixed armature. Preferably, the armatures are made from materials capable of being magnetised. The term armature used in this specification shall mean any apparatus of suitable material, capable of being treated, or capable of responding to a magnetic or other force field, and when placed in the vicinity of a magnet or other force, has its operational capability increased.

In earlier embodiments, the valve, referred to herein as the mag valve, comprised a mild steel bobbin with an attached seal, which was allowed to move on a horizontal plane when a surrounding coil member was energised, reversing the polarity of a rare earth magnet. The rare earth magnet provided energy to the valve, momentarily opening the seal between the valve and the plastic bulkhead, allowing fluid to travel through a narrow conduit to an outlet at the nose of the device. The metering valve system also included a coil member which is capable of being activated. On activation of the coil member, a solenoidal effect is produced which causes the moving armature to be attached to and seal against the stationary armature. Deactivation of the coil member enables the moving armature to move away from the fixed armature.

The term coil member used in this specification shall mean any apparatus capable of responding to an energy supply, such that a magnetic field, or other force field is created in the vicinity of the coil member. The coil member is a copper coil, which provides the energy to the magnet upon activation. The coil member is located around the chassis.

In preferred embodiments the structural configuration of the metering valve system creates a chamber within the conduit. Accordingly, movement of the moving armature towards and away from the fixed armature enables some of the substance which has passed into this chamber to move along the conduit to the outlet, thereby operating as a micropump.

Later more preferred embodiments, include a rare earth magnet and an actuator pin which opens the valve. These are also located within a chamber in the chassis. Both the magnet and actuator pin are placed within the chassis and then the filled progesterone reservoir is assembled to the chassis unit during the assembly process.

At the time of activation of the coil member, the polarity of the rare earth magnet is reversed, causing the magnet to strike the plastic actuator pin, which in turn opens the seal on the valve arrangement. The frequency of the activation of the coil member is controlled by the software program, as is the energizing duration. Fluid then travels past the actuator pin, down into the magnet chamber and, in turn, through a side-port out towards the outlet channel. The progesterone formulation is in contact with the magnet during delivery.

The actuator pin in the later preferred embodiments is made from Delrin 100 (a hard grade of acetyl) and is formed to suit the interior dimensions of the valve so that the valve flap is opened for only a specific amount at each stroke of the pin. The dimensions of the actuator pin against the interior dimensions of the valve and the distance that the pin travels are critical in determining the actual amount of fluid dosed at each stroke. The configuration of the pin can also be varied according to the amount of fluid to be dosed.

Therefore, variation of dosing can be achieved by varying a number of features of the device. Whilst some have been mentioned previously, such variations include:
a) Varying the actuator pin dimensions.
b) Varying the frequency of dose firing.
c) Varying the duration of opening.
d) Varying the outlet dimensions.
e) Varying the viscosity of the formulation.
f) Varying the spring energy available to the progesterone reservoir.
g) Varying the quantity of fill in the progesterone reservoir.
h) Varying the strength of the magnetic field surrounding the rare earth magnet.
i) Varying the strength of the rare earth magnet.

When the dimensions of the actuator pin are varied, the variation can be effected in two ways to affect the dosing of the formulation.

A variation can be also made to the length of the fine point of the pin, which strikes the seal. A longer pin length will open the seal further, which in turn causes more fluid to be dosed at each stroke of the pin. Alternatively, a change in the main body dimensions—for example, if the body of the pin is shortened—then the magnet has to travel further and there is a longer lag time in opening the valve. However, an actuator pin that is too short in the body will result in ineffectual operation of the valve.

Another feature of this later preferred embodiment is that the outlet is now located directly to the side of the valve arrangement (rather than at the nose cap end) and transfers the progesterone formulation from the valve to the outside of the device at the same position on the body as the valve is located.

The progesterone outlet is responsible for ensuring active delivery of the progesterone formulation to the target area within the body cavity. However, one of the important features of the progesterone delivery system is the outlet. The outlet features a reducing-diameter orifice, which in turn provides sufficient resistance to the progesterone fluid so that a positive “squirt of formulation” is achieved. By restricting—the end of the outlet tube the flow and quantity of progesterone formulation delivered at any one time is able to be governed more accurately. The restrictor effectively provides pressure to enable the flow of formulation to be managed. The preferred design has advantages over mechanical valves as the latter are typically unreliable at consistently producing small-dose outputs on an accurate basis.

As discussed previously, the progesterone valve was developed which allowed the outlet to be clipped through the body directly into the valve. The new progesterone outlet features a plastic injection-moulded component made from Delrin 100 (a hard grade of acetyl). The outlet features a series of reducing-diameter orifices, commencing at approximately 1 mm, reducing to approximately 0.7 mm and the further reducing to approximately 0.5 mm on the outside face. The diameters are reduced through a stage of three steps. The outlet is stepped to the final diameter of 0.5 mm to, again, provide back pressure on the valve—not for, the purposes of governing the valve operation but more for the purposes of providing a satisfactory squirt as the fluid leaves the outside of the device.

A further challenge when using the formulations delivered from the delivery device (and particularly with the progesterone formulation), was the generation of mucous in the anterior vagina.

Having the formulations, particularly the progesterone formulation in an alcohol solvent meant that when the formulation was delivered into the vaginal cavity, which is an aqueous environment, an immediate separation of the steroid from the alcohol occurred. In addition, significant generation of mucous is noticed. Tests on the mucous demonstrated high levels of progesterone being contained within the mucous. Accordingly, the progesterone would not be available for transfer through the vaginal mucosa. Effectively, the formulation in such cases fails to effect availability of progesterone to the vaginal mucosa. Therefore in effecting a reliable delivery mechanism for delivering progesterone formulation from the device, it was determined that the formulation required active expulsion and propulsion of the solution from the device in an attempt to clear the device and any mucous naturally accumulating around the device.

The small-diameter side outlet from the progesterone delivery apparatus whilst limiting the opportunity for animal fluid to travel into the valve via the outlet which could potentially comprise the integrity of dosing, also has a positive effect on mucous congregation. Animal studies revealed that side delivery of the progesterone formulation through this new outlet position was satisfactory at elevating necessary PPC levels and at the same time, that the change of position of the outlet from the front of the device to the side—of the device resulted in less mucous congregating around both the nose of the device and the progesterone outlet while in vivo. Further, water-soluble formulations, rather than alcohol based formulations have meant that excess mucous is no longer created.

This design of the delivery apparatus for the progesterone formulation, the smooth outside surface of the device, the improvements to the actual compounds in the formulation and changes to the amount of formulation being dosed, have all contributed to elimination of the occurrence of excessive mucous buildup around the outlet and other areas of the device per se.

A key objective of the delivery of progesterone from the device was that the active be expelled as far away as possible from the device to minimize the affects of the accumulation of mucous and to ensure the active was optimally presented in the cavity for transfer across the vaginal mucosa. The same objective is relevant also for the single-unit doses. Consequently, the single-unit doses features a plunger-type delivery system that, in turn, causes the contents of the single-unit doses to be expelled some distance and with some force from the device.

Again, a mechanical delivery system is adapted to deliver different formulations at known intervals into the animal to sustain a plasma concentration of the active in the animal, suitable for the breeding regime developed. The mechanical delivery systems of the invention are of course controlled by a complementary control system enabling variation in the delivery regime to be effected as required. For example, the pressure devices may be controlled by any controlling mechanism such as a micro-processor. However, the control system shall be described later.

According to another aspect of the present invention there is provided delivery apparatus for delivering a substance to an outlet of a substance delivery device substantially as described above wherein the delivery apparatus includes at least one conduit capable of containing the substance, at least one pressure device capable of applying pressure to the conduit at point(s) along the conduit, characterised by the application of pressure by the pressure device causing the substance within the conduit to move along the conduit to the outlet.

According to a further aspect of the present invention there is provided a method for delivering a substance to an outlet of a substance delivery device substantially as described above, including at least one conduit capable of containing the substance, at least one pressure device capable of applying pressure to the conduit at point(s) along the conduit characterised by the step of using the pressure device to apply pressure to the conduit causing the substance within the conduit to move along the conduit to the outlet.

According to a further aspect of the present invention there is provided a method of delivering a substance to a body characterised by the step of using delivery apparatus and/or method as previously described.

Single-unit dose delivery of any one or more of a tablet, a capsule, a liquid formulation from the front of the device was identified as a requirement of the present invention. The dose was required to be and adapted to be absorbed quickly and was delivered at a specific point in time. It was preferably not to be a prolonged delivery or a prolonged absorption of the active material.

Within preferred embodiments of the intra-vaginal delivery device used, the single unit dose actives are contained within conduit pots located in the front section of the device. However, in other embodiments, the single unit dose pots may be located anywhere in the device, as may be the liquid active reservoirs. Their location may be simply determined by the number and size of the various pots/conduits/reservoirs, and the preferred positioning of the outlets therefrom, which in turn may be dependent on the number of substances to be delivered and the size of the delivery device required to be accommodated in the particular cavity/passage of a particular animal or species.

In developing the delivery apparatus for the single unit doses, a number of issues were considered. For example, it was noted that liquid formulations had a greater tendency to leak from the pot system during transportation (particularly air transport, where pressure changes impacted negatively on the sealing ability of the liquid filled pots). Further, some slight movement of the nose cap was noted and the recommendation to develop a shipping cap or restraint as part of the delivery device packaging was identified. Improved seals and restraints were therefore used. However, development also focused on delivery of the single unit doses in substantially solid form (such as tablets, capsules, etc).

In developing the delivery apparatus issues considered were the ability to house a tablet in conduits within the delivery device; ability to release the contents to the cow; and the delivery means for doing so; and the ability to control the delivery means.

Accordingly, a number of nose pots incorporated into the delivery device design were developed that were capable of housing solid tablets, which were capable of delivering the tablets into the animal via a plunger system according to the product specifications and where the delivery could be controlled.

Therefore, in preferred embodiments described with reference to an example of controlling oestrus in cattle, doses of oestradiol and prostaglandin in individual solid capsules are contained within three single dose conduits/chambers located in the anterior of the delivery device. In some embodiments of the present invention the number and length of the conduit(s) containing the unit doses may vary, but there is typically at least one pressure device associated with each conduit.

In preferred embodiments the release mechanism for delivery of the unit doses includes a plunger that, when fired, fully extends to the front of the device (flush with the front of the chassis). A significant number of iterations were developed so that the design of the plunger operates with the specific unit doses that have been or will be developed. Specifically, consideration has had to be made for stretch of the plunger over time due to the spring tension placed on the nylon material from which the plunger is manufactured, structural thickness and strength so that the plunger does not exit the device after firing, and that minimal opportunity exists for fluid ingress into the device due to the shape and design of the plunger. To this end, the head of the plunger has been further modified to provide a close tolerance, but not snug, fit to the chassis wall, minimising the amount of animal fluid that may ingress while in situ.

The plunger is preferably held under tension in its pre-delivery position. The plunger mechanism is preferably released via heat applied to a portion of the plunger design. The heat effectively melts the portion of the plunger and releases it. A spring associated with the plunger propels the plunger along the conduit and applies pressure to the unit dose causing it to be expelled under force from the unit dose pots.

Energy required to heat and ultimately melt the plunger stem thereby enabling its operation, is preferably provided by energy sources within the substance delivery device. In preferred embodiments, the energy source is provided by at least one battery. However, other embodiments may use energy sources in the surrounding environment. The energy sources may include kinetic, chemical, or thermal energy. For example, the internal temperature of an animal is 37° C. and such temperature (heat energy) may used as an energy source itself or be converted to kinetic energy, such as for effecting or triggering operation of systems including heat sensitive components.

In preferred embodiments, a thin, nylon tail of approximately 0.8 mm in diameter has been fitted to the tail of the plunger to allow assembly workers to grasp the plunger without damaging the plunger stem as previously damage had been incurred to the plunger during assembly through handling of the item.

Further, effective sealing of the electronics chamber has been completed through modification to the O-ring area, which in turn has resulted in the product being more robust during air transportation. For example, there is no significant expansion of air within the device during transportation and there is no longer a cause-effect relating to premature opening of nose caps during air transport. This has also been assisted with a move from a liquid formulation to a tablet formulation.

The specifics of the plunger dimensions, the number of unit doses that can be delivered and the timing of delivery are all relevant to the requirements of the product. In other words, more than one unit dose can be delivered or as many unit doses can be delivered as is physically possible.

Each individual unit dose chamber is preferably closed in a substantially fluid tight manner via a nose cap. The nose cap is preferably resiliently hinged to the body of the nose of the device, at at least one location around its perimeter. Santoprene™ is the preferred material for the nose cap. Santoprene has been chosen because it has some compressibility when the nose cap is closed and fitted to the unit-dose chamber. It also has the ability to be elastic so that, when the plunger system is fired, a hinge on one side of each individual nose cap retains the nose cap and prevents inadvertent loss. The nose cap provides a snug push-fit to each of the unit-dose chambers and provides now an effective seal against the chassis reservoir.

The pressure devices which operate as described above are either capable of controlling the flow of substance through the conduit, and/or capable of controlling release of the substance from the conduit. Conventionally, conduits require valves to prevent back flow. The capability of the pressure devices of the present invention to control the flow and/or release of substance removes any need for separate valves to prevent such back flow.

It can be seen that the present invention has a number of advantages over the prior art. The pressure devices are small, simple, and require little maintenance and at least three are able to fit inside the body of the delivery device. Further, the pressure devices are capable of at least effecting release of more than one substance, in more than one form, at more than one time into the animal.

Whilst the release of progesterone has been previously described with reference to release of a liquid formulation from a collapsible bellows, it should be appreciated that in yet further preferred embodiments the progesterone may be released from conduits substantially similar to, if not the same as, the unit dose delivery system. Given the challenge of delivering progesterone (as may apply to any other relevant substance) requires more frequent delivery, both the method of operation of the unit dose system and the formulation itself may be adapted as required. For example, the progesterone may be in substantially solid form and may rely on body fluids along with the processes of dissolution and osmosis to effect substantially continuous delivery of the progesterone passively in to the environment around the substance delivery device. A plunger and/or spring system may operate substantially from the base of the conduit to urge the solid form of progesterone along the conduit to the outlet orifice as the surface of the solid form is eroded away. The outlet may be positioned on any effective surface of the device to effect optimum delivery of the substance.

The substance may be presented in the conduit as a series of stackable tablets, or as a single block. The rate of dissolution may be controlled passively through the use of dissolution enhancing or dissolution limiting substances incorporated within the tablets or solid block at varying locations.

This passive delivery using a solid form of the substance may be used alone or in conjunction with a simultaneous, continuous and/or periodic release of a substantially fluid form of the same substance, as may be required to effect the desired outcome.

According to another aspect of the present invention there is provided a method of introducing a substance into an animal from a substance delivery device substantially as described above, characterised by the step of controlling the operation of the delivery apparatus so that the substance is actively introduced into the animal.

Many biological functions are based on hormonal interactions involving the integrated responses of a number of substances, as in the case of the oestrus cycle. The preferred delivery device is effective in controlling the time of delivery of more than one substance, as well as the volume delivered on a dose by dose basis.

The control means effecting control of delivery is an electrical control system. The control means not only controls delivery of the various active ingredients/formulations from the delivery device by instructing the delivery apparatus to release the formulations into the animal according to a predetermined specification, but triggers operation of the device per se, and is able to be reprogrammed to effect recalibration of the delivery apparatus to further regulate delivery of the formulations.

The control of delivery is gained through specialist dose control software that is programmed into a microchip and provides the instructions for the device and determines when the delivery activities will occur. The microchip is energised by a miniature power cell (which provides energy so that the activities can occur) and controls operation through a printed circuit board and its components and electromagnetic coil, and the on/off switch (which activates the device and determines the time of the delivery process commencing).

The on/off system control means is always related to the printed circuit board and the activation of the printed circuit board. That is, before the device will operate the circuit board must be activated, either from a shutdown or a standby mode.

In one preferred embodiment an integral on/off switch is located on the top side of the circuit board. The device may be activated manually at the time of insertion of the device by depressing the switch manually for approximately two seconds. Depression of the switch by the user, is effected through a soft, plastic membrane on the outside of the body. When the on/off switch is depressed for the required time, an LED will flash, confirming operation. The software utilised in the current version allows the operator to turn the device off by, again, depressing the on/off switch for a pre-determined time period.

However, in other embodiments the device may be activated from a remote source, by electronic signaling means. For example, the device may remain inactive within the animal for a period of time until the farmer wishes to start the fertility cycle. At which stage, the farmer may have an external device to the animal which triggers the action of the delivery apparatus or wakes up the microprocessor. This external device may come in many forms, for example it may be a radio transmitter, an ultrasonic transmitter, a magnet and so forth. Similarly, the device may also be controlled, triggered and programmed from an external source.

Preferably, the control means features a printed circuit board with relevant components. The integrated circuit board contributes to the operation of the control sequence. During development of the circuit board a number of layout and component changes have been undertaken, with a number of microchip and surface-mount component changes being evaluated and tested. The printed circuit board is assembled as an integral unit and clips directly into the chassis.

The circuit board is preferably fitted to the chassis. The method of fixing the circuit board to the chassis has been improved and modified over the development process and includes a strengthened bulkhead at the front end of the chassis.

The circuit board also receives portions of the delivery apparatus for the unit dose pots. Positive alignment of the plunger straps of the unit dose pot delivery apparatus is effected over the resistors on the front edge of the circuit board. As the resistors heat up, they cause the plunger straps to melt and effect operation of the delivery apparatus to deliver the appropriate unit dose into the animal.

The circuit board also includes welded tabs from either end of the power cell that are soldered to the circuit board.

The control means also includes a microchip that has software specifically developed to regulate the time and operation and triggering of the delivery apparatus for specific dosing regimes. In preferred embodiments of the present invention the microchip is capable of being reprogrammed as required to reflect recalibration of the dose regime thereby actually altering the size of the dose as well as the timing and duration of the dose depending upon the environment the device is in.

In some embodiments of the present invention, remote programming of the microprocessor may be possible from outside the animal, or at time of insertion. In other embodiments of the present invention the microprocessor may be able to communicate to an external device. For example, the microprocessor may feed back data as to how much substance has been delivered, the temperature of the environment and so forth. An appropriate microprocessor for use with the present invention is a four bit microprocessor or an 8 bit single chip microprocessor such as a pic 16c54 or Z8 or Motorola 6805

The control means is also powered by an energy source. In preferred embodiments, the control mechanism is powered by an independent power cell that provides energy to the circuit board. The power cell preferably used is a 3v specialist lithium power cell, which features welded tabs on either end that are in turn soldered to the circuit board.

According to a further aspect of the present invention there is provided a method of controlling the delivery apparatus of a substance delivery device substantially as described previously characterised by the step of introducing predetermined amounts of at least one substance at predetermined times into the body of an animal.

The advantages of having active control over the introduction of the substance is readily apparent. For example, where the pressure devices are actively operated by the control means the rate and volume of a substance being delivered may be controlled. Accordingly, active delivery means that there is greater control so that the precise concentration of the substance can be delivered at the precise time, independent of the environment surrounding the substance delivery device, or delivery apparatus.

The means by which the microprocessor can control the pumps/pressure devices of the delivery apparatus can vary. The pump may be powered by an energy source (perhaps a battery, an external source such as the animal's natural body temperature, a magnetic field, a spring or the like) and the action of the microprocessor may be merely to connect or disconnect the pumps from the energy source. Alternatively, the microprocessor may control the valve or valves which permit or prevent the flow of substance from the delivery apparatus, and substance delivery device.

Accordingly, the delivery regime can follow any sequence depending on the dose control software programmed into the microchip. With the preferred delivery device, dose control is exerted over the time at which a unit dose is delivered (for oestradiol and prostaglandin unit doses), and over the timing and dose volume of a continuous series of pulsatile doses (for progesterone doses).

In one embodiment of the present invention, the microprocessor may be programmed to control the release of varying doses of differing hormones into the animal at predetermined times, thus allowing for accurate determination of when oestrus occurs. For example, the release mechanism for firing the front single-unit doses is located at the front of the circuit board. Therefore, the control mechanism becomes part of the delivery system in that the release mechanism for the single-unit doses is contained on the circuit board. At an appropriate time, the control mechanism activates the power (electricity) to the relevant point on the circuit board that, in turn, releases the single-unit dose. With regard to the progesterone delivery, similarly, the control mechanism energises the progesterone delivery system by sending an electrical current to the pump, valve or Piezo element.

There are further advantages of having an active control delivery of substance to an animal. For example, there may be provided sensors which monitor the environment around the substance delivery device or delivery apparatus. The sensors may determine when the environment is ideal for the introduction of a substance into the body of the animal. This information may be then acted upon by the microprocessor to control the delivery apparatus to introduce those substances.

For example, the sensors could determine factors in the body fluid surrounding the substance delivery device and/or delivery apparatus, such as temperature, acidity, viscosity or even odour. These physiological indicators may in some instances be more accurate than a calendar date for determining when certain substances should be introduced into the animal. With active control, an accurate response to these physiological conditions is possible.

It should be appreciated that although reference throughout this specification has been made to the use of the present invention as a substance delivery device or delivery apparatus for use within animals, the delivery apparatus, control mechanisms, and retention apparatus described may be used in substance delivery devices in other environments, particularly in environments which it is not possible to directly access the substance delivery device.

### BRIEF DESCRIPTION OF DRAWINGS

Further aspects of the present invention will become apparent from the ensuing description which is given by way of example only and with reference to the accompanying drawings in which:

FIG. 1 is a diagrammatic perspective view of a retention apparatus in accordance with one embodiment of the present invention, and

FIG. 2 is a diagrammatic cross-sectional perspective view of a retention apparatus in accordance with one embodiment of the present invention, and

FIG. 3 is a diagrammatic perspective view of the nose cap in accordance with an embodiment of the present invention, and

FIG. 4 is a diagrammatic cross section of the front portion of the body in the region of the nose cone, showing locking feature for the chassis, in accordance with an embodiment of the present invention, and

FIG. 4b is a diagrammatic side view of the nose cone in accordance with the embodiment of the present invention in FIG. 4a, and

FIG. 5 is a diagrammatic side view of the front portion of the body in accordance with an embodiment of the present invention, and

FIG. 6 is a diagrammatic perspective view of the rear portion of the body in accordance with an embodiment of the present invention, and

FIG. 7 is a diagrammatic side view of the chassis in accordance with an embodiment of the present invention, and

FIG. 7a is a diagrammatic anterior end view of the chassis of FIG. 7 in accordance with that embodiment of the present invention, and

FIG. 7b is a diagrammatic cross-sectional view of the chassis in the region of the single unit pot conduits in accordance with the embodiment of the present invention in FIGS. 7 and 8a, and

FIG. 8 is a diagrammatic perspective view of a plunger in accordance with an embodiment of the present invention, and

FIG. 9 is a diagrammatic side view of the bellows in accordance with an embodiment of the present invention, and

FIG. 9a is a diagrammatic perspective view of the anterior end of the bellows in accordance with the embodiment of the present invention in FIG. 10, and

FIG. 9b is a diagrammatic perspective view of the rear end of bellows in accordance with the embodiment of the present invention in Figure, and

FIG. 10 is a diagrammatic side view of the actuator pin used in accordance with one embodiment of the present invention, and

FIG. 11 is a diagrammatic cross-sectional view of the restrictor outlet from the progesterone bellows/valve in accordance with a preferred embodiment of the present invention, and

FIG. 12 is a diagrammatic rear view of the valve used in accordance with the progesterone delivery apparatus of a preferred embodiment of the present invention, and

FIG. 12a is a diagrammatic side view of the valve used in accordance with the progesterone delivery apparatus of a preferred embodiment of the present invention, and

FIG. 12b is a diagrammatic cross-sectional view of the valve used in accordance with the progesterone delivery apparatus of a preferred embodiment of the present invention, and

FIG. 13 is a diagrammatic front view of a flap valve of a progesterone delivery apparatus used in accordance with a preferred embodiment of the present invention, and

FIG. 14 is a diagrammatic side view of a valve ring of a progesterone delivery apparatus used in accordance with a preferred embodiment of the present invention, and

FIG. 14a is a diagrammatic cross-sectional view of a valve ring of a progesterone delivery apparatus used in accordance with a preferred embodiment of the present invention, and

FIG. 15 is a diagrammatic side view of a tail rod for use with retention apparatus used in accordance with a preferred embodiment of the present invention, and

FIG. 16 is a diagrammatic plan view of a strap and patch retention apparatus used in accordance with a preferred embodiment of the present invention.

### BEST MODES FOR CARRYING OUT THE INVENTION

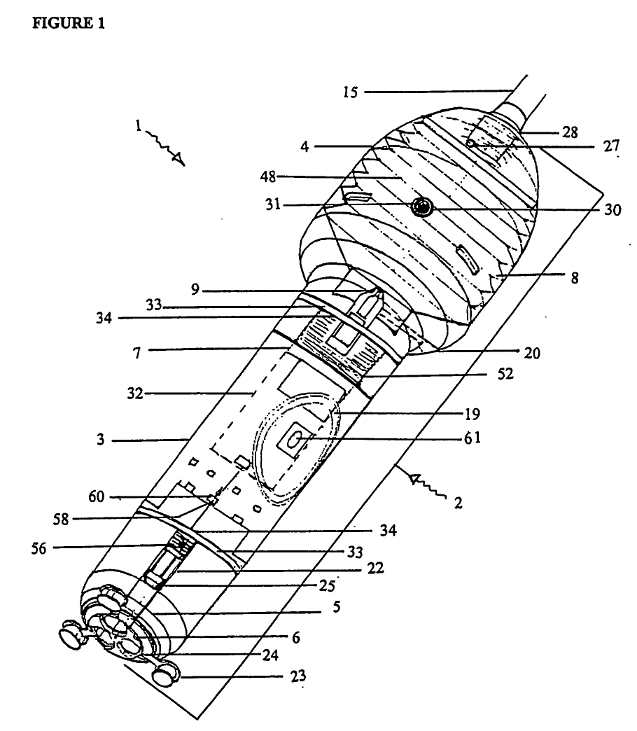

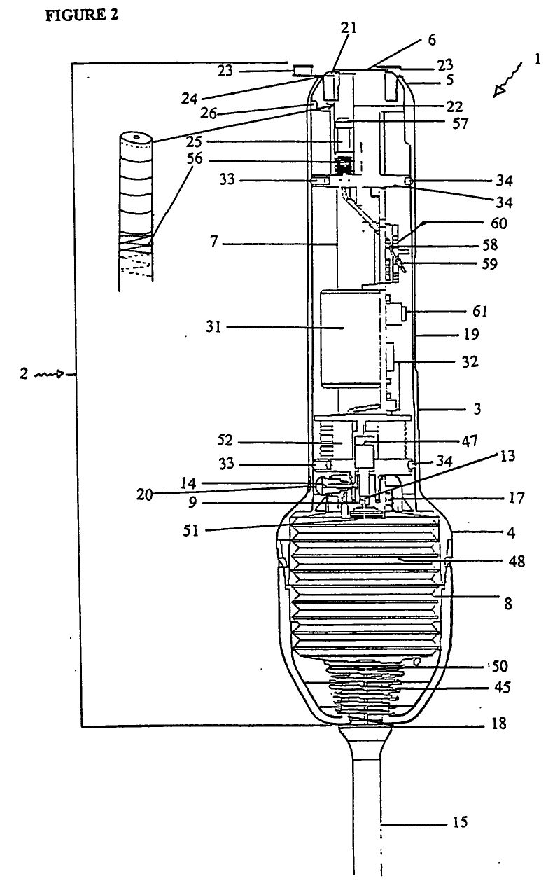

With reference to the diagrams by way of example only there is provided substance delivery device generally indicated by arrow 1. The substance delivery device 1 is capable of insertion into a body cavity or passage of an animal. FIGS. 1 and 2 are diagrammatic perspective views of the device 1.

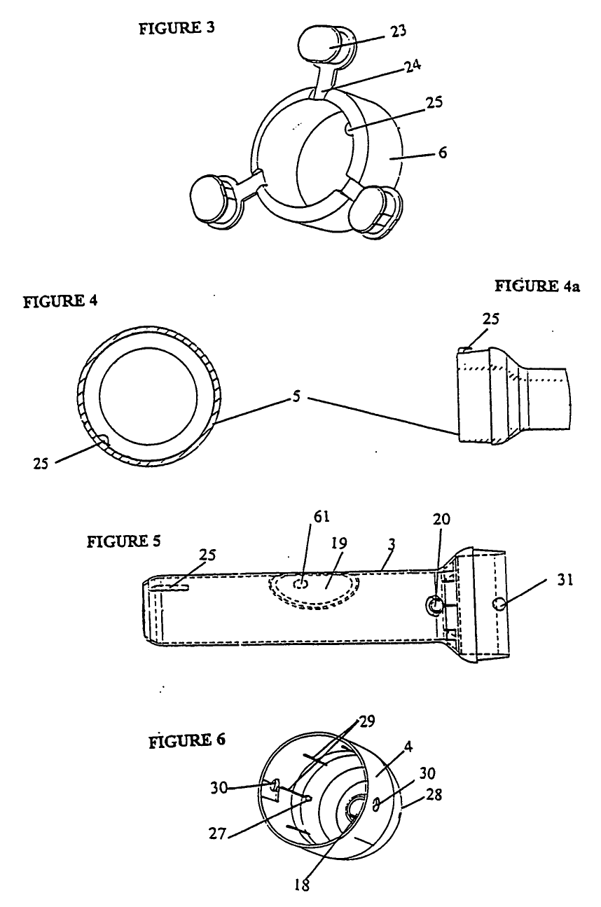

The substance delivery device 1 includes a body generally indicated by arrow 2. The body 2 includes a front portion 3 (shown in FIG. 5), a back portion 4 (shown in FIG. 6) and a nosecone arrangement 5 associated with the front portion (shown in FIGS. 4 and 4a). The nose cone portion 5 interacts with nose cap 6 (shown in FIG. 3).

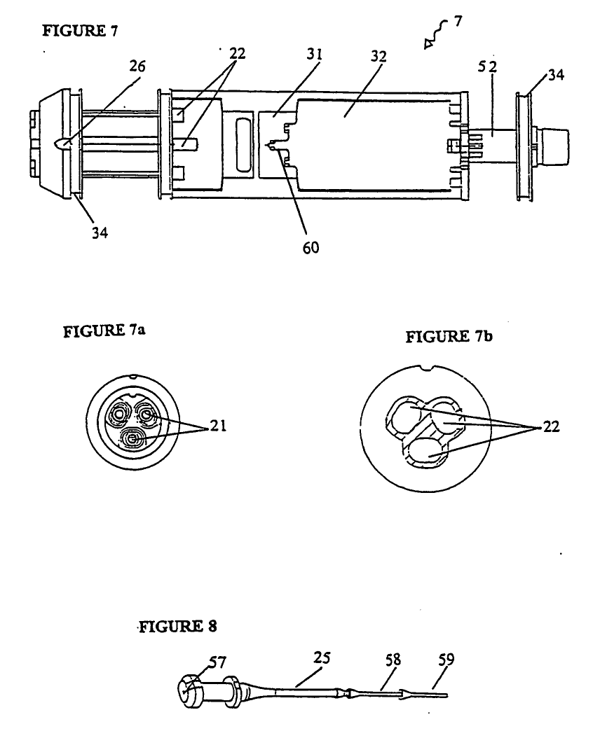

The body 2 encases the chassis generally indicated by arrow 7 (shown in FIGS. 7, 7a and 7b) of the device 1 which is essentially the skeleton of the device 1 and with which most of the internal components are associated.

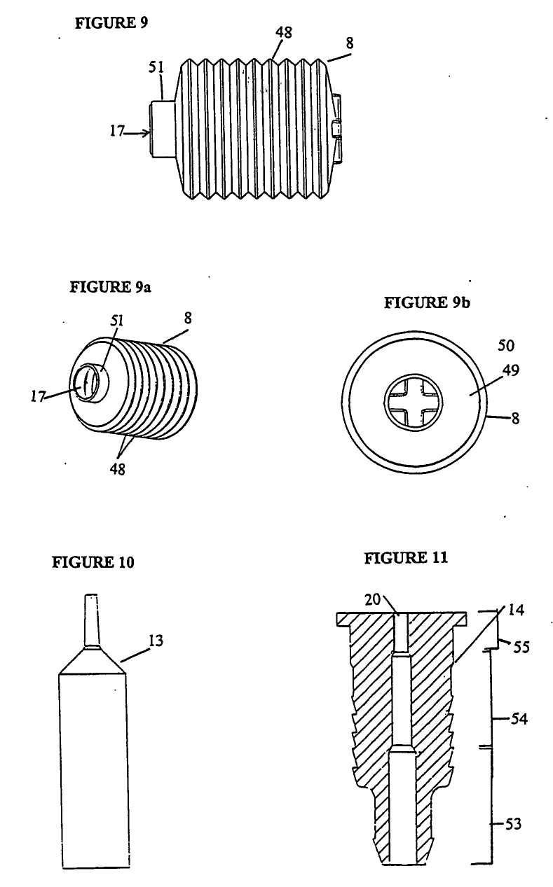

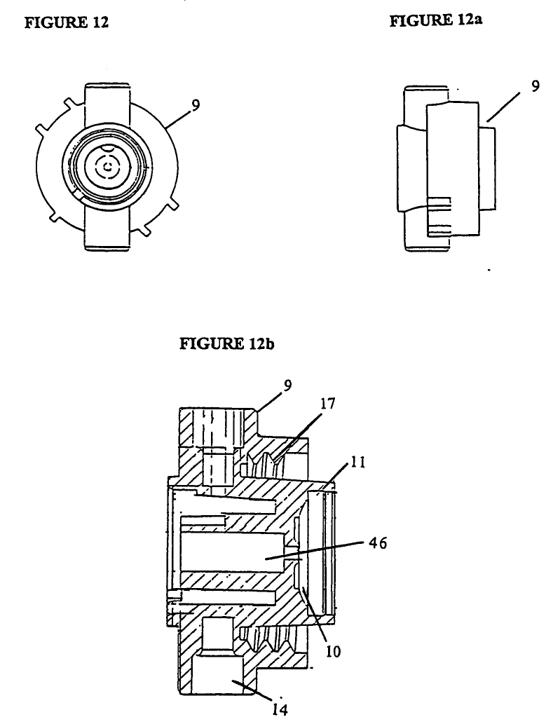

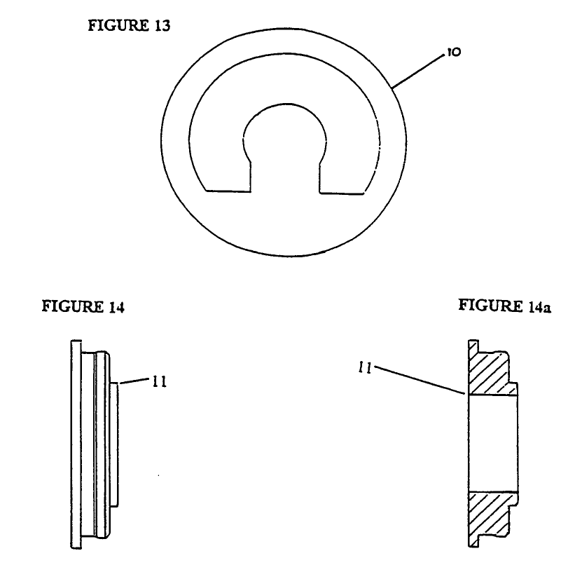

The rear portion 4 of the body 2 encases a reservoir 8 (or bellows shown in FIGS. 9, 9a, and 9b) which contains a liquid formulation. The bellows 8 is screw-threaded at 17 to a valve 9 (shown in FIGS. 12, 12a and 12b). The valve 9 includes a valve seal 10 (shown in FIG. 13) and a valve ring 11 (shown in FIGS. 13 and 13a). Release of formulation from the bellows is effected by a striking action of an actuator pin 13 (shown in FIG. 10). Formulation is released from the device via the action of the valve 9, through an outlet channel 14 (shown in FIG. 11) and into the animal's cavity.

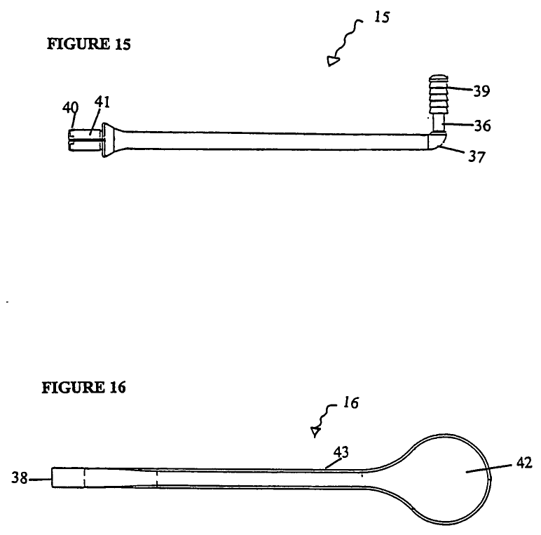

The device 1 also includes a tail rod 15 (shown in FIG. 15) which is inter-connectable with external retention apparatus 16 (shown in FIG. 16).

The substance delivery device is designed to autonomously deliver in situ one or more substances, in either or both solid and fluid forms, from separate reservoirs via substantially dedicated outlets, to an animal. The substance delivery device includes delivery apparatus and programmable control means for effecting controlled delivery of each substance from the device, in accordance with a preferred delivery regime. Accordingly, the substance(s) are delivered to the animal in predetermined quantities, for predetermined dose duration, at predetermined times to effect desired concentrations of the substance in the animal as required to effect a desired outcome. The tail rod 15 provides means for attachment of the retention apparatus to maintain the device in situ during the period over which the device is required to deliver the substance(s) from the substance delivery device.

A more detailed description in relation to the figures now follows.

The body 2 is a substantially hollow receptacle including a chamber capable of housing the chassis 7 of the device 1 along with the delivery apparatus and associated controlling apparatus (including a power source as required) of the device. The body 2 is further adapted at 18 to receive the attachment means or tail rod 15 for the appropriate retention apparatus 16 used with the device.

The function of the body is to protect the internal components and formulations from animal fluids and also to protect the device during transportation and handling prior to insertion.

The body in the illustrated embodiments of FIGS. 1, 2, 5 and 6 includes at least two major portions, such as the front portion 3 and the rear portion 4 which are inter-connectable. The front portion of the body includes an integral nose cone 5. However, this may be a separate portion in other embodiments. The front portion 3 is typically the most anterior, or leading portion when the device is inserted into the animal. The rear portion 4 is located at the trailing end of the device.

The front portion is substantially elongate and cylindrical in shape, having a substantially uniform cross-sectional profile. The rear portion is a substantially larger-diameter, ovoid cylinder, being substantially shorter than the front portion.

The front body portion, includes a substantially thinner and includes an indented portion 19. This indented portion coincides with the location of the on/off switch (shown in FIGS. 1 and 2) of one embodiment, to allow an operator to be able to activate the on/off switch of the control means of device externally by depressing the switch through the thinner body wall before the device is inserted into the animal. The thinner walled portion also improves visibility of an indicator light (not shown) that indicates when the device is activated.

Towards the rear end of the front portion of the body at least one orifices/apertures 20 is included. This is one of the outlet(s) from which the substances in the reservoir(s) are delivered. For example, in FIG. 5 the embodiment illustrated includes an aperture 20 where the outlet channel 14 (as shown in FIG. 11) for a progesterone reservoir is located, even though the reservoir for this substance may not necessarily be encased by the front body portion. In the embodiment described, the progesterone reservoir, or bellows 8, is located towards the rear of the fully assembled device, yet the outlet pin is located flush against the outside surface of the front body portion.

The front portion also receives a nose cap portion 6 (as shown in FIG. 3) substantially at the leading distal end. The nose cap portion communicates with outlet orifices 21 associated with conduits/reservoirs 22 (as shown in FIGS. 7 to 7b) dedicated to containing separate unit-dose substances. The cap portion includes removable sealing means, or caps, 23, which are hinged at 24 to enable the caps to be opened for delivery of substances contained in the reservoirs, when required in accordance with the preferred delivery regime. When sealed, the caps 23 provide a substantially fluid tight seal to each of the substance reservoirs 22.

The substances are expelled from the reservoirs/conduits by the controlled operation of pressure devices 25 (one of which is shown in FIG. 8) associated with each reservoir 22. The delivery process causes release of the removable sealing means/cap 23, allowing the contents of the substance reservoirs to be delivered into the animal. The reservoirs/conduits 22 are associated with the internal chassis portion 7 of the device 1, and in the illustrated embodiment are enclosed within the front body portion 3.

The front portions (3,5 and 6) of the body locate the position of the internal chassis of the device, via a slot 25 (shown in FIGS. 5, 4a, 4b and 3, respectively) cut into the body which matches to a corresponding complementary configured ridge 26 located on the chassis (as shown in FIG. 7).

The rear body portion 4 or rear cap (shown in FIG. 6), as it may also be called, in the embodiment being described houses a liquid progesterone formulation reservoir, or bellows 8 (shown in FIGS. 9 to 9b). Although, in other embodiments, more reservoirs may be located in this region. The bellows is a collapsible reservoir.

The rear body portion also features a breather hole 27 in the area relating to the progesterone reservoir to allow the air pressure within the device to equilibrate as the progesterone reservoir collapses as it is emptied during dosing.

The rear portion, also includes a recess 18 on the horizontal access at the rear 28 of the rear body portion, for insertion of the tail rod 15. The tail rod is force-fitted to the rear body cap during assembly. The tail rod provides a secure anchor point for attachment of the retention system to the rest of the device. The inside of the rear body cap may be strengthened with a series of plastic ridges 29 or such like to provide more rigidity to the rear portion to support both large fluid filled reservoir(s) and/or the tail structure.

The rear body cap features a locking system to the front body via a number of recesses 30 moulded into the rear body portion. These recesses are capable of receiving and interlocking with complementary configured barbs/projections 31 on the front body portion (shown in FIG. 5). The connection area between the two body components is strengthened so that when the two halves are married together and joined as a press-fit, the two parts are preferably inseparable. It is important that the device does not fall apart whilst inside an animal.

The outside of both body portions has a very smooth finish. The smooth finish along with the soft plastic body and the cylindrical shape are designed to improve the ease with which the substance delivery device is able to be inserted into and withdrawn from a passage or body cavity of an animal, with minimal difficulty, without effecting damage and/or discomfort to the animal.

The body portions lock over the chassis to protect the chassis and its internal components from animal fluids. The chassis of the device is the skeleton of the product. From the chassis, all of the other components are attached.

As previously mentioned, the chassis (shown in FIGS. 2 and 7) includes up to, or more than three front conduits/reservoirs 22, but also includes a power source (battery) 31, a printed circuit board 32, the valve 9 (for liquid progesterone delivery), as well as the pressure devices 25 for the front reservoirs. The valve 9 for the liquid progesterone reservoir located at the rear distal end of the chassis.

The chassis is retained in place in the body via locking lugs (previously discussed) maintained in a fluid tight manner against the front body section via O-rings 33, positioned in appropriate grooves 34 (as shown in FIGS. 2 and 7).

The O-rings effect a fluid-tight seal for the internal electronics particularly where there are venting holes (which allow animal fluids into the device) to allow atmospheric pressure to equilibrate during progesterone dosing. The O-rings are located around the chassis and press against the body.

In the illustrated embodiment the chassis is approximately 116 mm in length and has a maximum radius of 22.6 mm and is manufactured from polypropylene plastic.

The tail rod 15 of the device is the portion of the device that contributes both in part to insertion of the device into an animal, as well as to retention of the device within the animal's cavity (where the cavity is the vagina). The tail extends from the rear of the body of the device to the exterior of the aninal's vulval lips. In effect, the tail comprises an internal connection between the retention apparatus and the device. An anterior portion 40 of the tail rod is rigidly fixed into the body of the device, such that when the tail rod is connected to the retention apparatus the orientation of the tail is pre-determined and causes the device to have the same orientation within the animal in all instances.

The tail includes a substantially elongate, substantially straight, internal shaft portion attached to the rear portion of the body and an external retention apparatus retaining portion (located at the outer distal end of the tail). The retention apparatus retaining portion is substantially angled with respect to the shaft portion and is adapted to receive a complementary configured attachment portion 38 of the retention apparatus 16.

The shaft has a substantially limited, substantially circular, cross-sectional dimension to enable retention of the device without impeding the flow of body secretions necessary for the normal biological functioning of an animal's reproductive system, or causing discomfort or injury to the animal.

Accordingly, in the illustrated embodiment, the tail protrudes from the rear of the body (once fitted) by a distance of approximately 126 mm. The tail rod is substantially circular in a cross-section and is approximately 14 mm in diameter along its length. The distal portion is angled at right-angles to the shaft and extends for approximately 12 mm prior to the commencement of the barbs 39 where the external retention apparatus is attached. The location of the right-angled distal portion determines the depth of insertion as this right angle is placed against the vulval lips of the animal.

In the embodiment illustrated in FIGS. 1 and 15 the tail is secured to the body via a number of plastic barbs 41 around its anterior portion 40, which positively engage with the softer material of the body when the rod is press-fitted into the aperture in the rear portion of the body.

The exterior surface of the tail is preferably smooth so as not to cause any abrasion or trauma with the target animal. The smooth surface of the tail also ensures that any animal excrement, mucous or body fluid does not bond or bind to the tail once inserted and while in situ.

The tail in preferred embodiments is manufactured from Du Pont Zytel 331, which is a 33% glass, reinforced with nylon 66%. It is a very rigid, non-flexible material. Any flexibility may negatively impact on the ability of the tail to be used in the insertion of the device, or may jeopardise the retention of the device in the animal for the length of time required to effect delivery of the substances into the animal. However, any suitable material may be used for the tail provided the material enables the tail to function as required.

The rear distal end of the tail rod of the illustrated embodiment includes a series of protrusions/barbs 39 for attachment of the external retention apparatus 16. Where the retention apparatus is made from rubber, the elastic nature of the rubber, enables a cavity 38 in the end of the rubber to fit over the rod and the hard nature of the rod material against the soft pliant rubber, ensures a non-removable fit.

The retention apparatus is attachable at the rear distal end of the device. This enables the retention apparatus to be attached in appropriate positions on the outside of the body of the animal as required to enable the device to be more reliably retained within the animal's body cavity. The positioning of the retention device is largely dependent on the animal's behavioural characteristics. For example, naturally passive animals, such as cows may allow the retention apparatus to be attached to the back of the animal. However, naturally inquisitive animals, such as pigs may easily pull on and remove retention apparatus attached in such locations. Therefore, it may be necessary to ensure the retention apparatus surrounds only the vicinity of the vulval area, relying on the animal's reluctance to allow other animals to touch that area to minimize the likelihood of another animal pulling off, or chewing the retention apparatus.

In the illustrated embodiment, the retention apparatus is substantially elastic/resilient and comprises a substantially circular/ovoid patch 42 and a substantially elongate strap 43 arrangement. The patch is preferably approximately 55-60 mm in diameter and is attached to the back of the animal by suitable adhesive, or other means. The strap attached to the tail rod and is retained under sufficient tension to maintain the device inside the animal. In other embodiments, the retention apparatus may include a cap, diaphragm or similar means that applies suction or adhesion around the vulval region and retains the device in situ.

Having the retention apparatus attached externally of the animal obviates problems associated with the retention apparatus increasing or impeding the flow of body secretions, such as intravaginal mucus, or causing discomfort to the animal or causing damage to the interior walls of the cavity (the vagina) during insertion, retention or withdrawal of the device.

The retention apparatus is preferably made from a material which is capable of being moulded, capable of being sterilised for hygienic reasons, is lightweight, chemically resistant, can withstand wet environments, and is economical.

To effect the treatment regime for the delivery device required to synchronise/control/regulate oestrus, four hormone formulations need to be delivered at strategic times. The drug regime therefore developed involves delivery of two formulations of oestradiol, one of prostaglandin and a progesterone formulation over a duration of time. The design concept for the delivery device includes controlled timed delivery of single-unit doses for some hormones and continuous dosing of others, to replicate the naturally occurring situation in the animal.

To effect continuous, pulsatile dosing of progesterone the hormone is provided in solution and retained within the collapsible bellows 8. To deliver very, very small quantities of progesterone formulation required per dose to effect the desired blood serum levels over the progesterone delivery period, the delivery system is required to be very precise and able to be monitored, measured and accurately calibrated/re-calibrated. The pressure/force applied to the substance(s) being delivered from the substance delivery device is also required to be an expulsive force.

The progesterone is preferably dissolved in solution. Preferred solvents such as Marlophen NP3 and/or Propylene glycol P1000 and/or 2-Phenylethanol may be used, although ethanol and water are also suitable solvents. Use of cyclodextrins may further improve the solubility of the progesterone solution.

The design concept for the delivery of progesterone requires pressure to be applied to the back of a collapsible reservoir by a seal 44 and spring 45 (shown in FIG. 2). The actuator pin of the valve 9 is then opened for a specific period of time to allow some of the contents of the reservoir to fill a small delivery cavity 46 (shown in FIGS. 2 and 12b). Pressure from repeated operation of the valve 9 effectively drives the fluid from the device to the outlet hole 20.

The bellows (reservoir) is firstly evacuated under vacuum and then filled with the appropriate quantity of progesterone formulation. It is maintained under positive pressure by means of a spring 45 force applied to the back end. Due to the positive pressure, the fluid is always presented to the inlet 46 of an associated micro-pump to which the reservoir is attached regardless of the attitude of the device. More specifically, the micropump is operated by means of a magnetic core 47 that is forced to stroke when the electromagnetic coil, within which it is housed, is energised. The core activates a release mechanism, or actuator pin 13, that allows a predetermined quantity of solution to enter the micropump. The positive pressure in the reservoir ensures the solution enters the micropump and is expelled from the outlet 20 under pressure. The micropump is very energy efficient to enable small low energy power cells 31 to be used.

In the illustrated embodiment of FIGS. 9 to 9b, the progesterone bellows is a separate component to the chassis. In other embodiments the progesterone reservoir/bellows may be an integral part of the chassis. Polyethylene has been selected as a material to use in the bellows, which features a number of annular rings 48 enabling the bellows to collapse in concertina fashion due to spring pressure directed at the rear end 49 of the bellows. This directs fluid contained within the reservoir out, to be expelled. The rear end 49 of the bellows reservoir is shaped and strengthened with a cross or supporting structure 50 to provide even collapsing of the bellows. Various models have been developed, ranging in capacity from 12 ml to 42 ml. In larger diameter bellows arrangements, a seal guide type configuration may effect even transfer the energy from the spring to the bellows unit to minimise racking and implosion of the bellows. In such embodiments, seal guides and silicone lubricants may also be used.

The bellows in the illustrated embodiment is open-ended at its anterior end 51. As the bellows collapses fluid contained therein is directed towards the anterior end of the bellows. A valve 9 is located at the anterior end of the bellows. The bellows is able to couple directly to the valve via a screw-thread at 17, which provides the means of attachment to a valve. The portion of the bellows unit at the valve end of the bellows is tapered to the point where it joins to the valve to ensure the maximum possible amount of progesterone formulation can be delivered to the valve.

With this metering valve system the spring tension helps to ensure that the valve system is able to reliably seal, when required.

The bellows valve (or progesterone valve for the purpose of this exampled description) comprises a number of fixed and moving components, both metallic and plastic, that are fitted together to form the valve. The valve 9 is responsible for sealing the progesterone reservoir and preventing inadvertent release of the formulation to the outlet as well as allowing the progesterone formulation to be released according to the specification.

The valve 9 features a small hole in the very center, with a sealing face. Against the sealing face, a silicone flap 10 (as shown in FIG. 13) is positioned. A valve ring 11 (as shown in FIGS. 14, 14a) is applied over the silicone flap 10 to lock it into place. This arrangement is then threaded onto the bellows, which results in the bellows being an integral reservoir.

The valve 9 of the illustrated embodiment operates by spring energy transferred against the bellows reservoir causing the progesterone fluid to press against the silicone seal. This keeps the valve closed at all times while the valve is not being operated.

The valve operates on a reversed magnetic polarity principle, and includes a moving and a fixed armature. Preferably, the armatures are made from materials capable of being magnetised. A copper coil member provides energy to the magnet upon activation. The coil member is located around the chassis at 52. A rare earth magnet and an actuator pin 13 which opens the valve are also located relative to the chassis. Both the magnet and actuator pin are placed within the chassis and then the filled progesterone reservoir is assembled to the chassis unit during the assembly process.

At the time of activation of the coil member, the polarity of the rare earth magnet 47 is reversed, causing the magnet to strike the plastic actuator pin, which in turn opens the seal on the valve arrangement. The coil member is operated at a low frequency and at a very low duty cycle. Typically however, the on off times of the coil member will be greater than one second. The frequency of the activation of the coil member is controlled by a software program (specifically designed for use with the invention), as is the energizing duration. Fluid then travels past the actuator pin, down into the magnet chamber and, in turn, through a side-port out towards the outlet channel 14. The progesterone formulation is in contact with the magnet during delivery.

The actuator pin 13 is made from Delrin 100™ (a hard grade of acetyl) and is formed to suit the interior dimensions of the valve so that the valve flap is opened for only a specific amount at each stroke of the pin. The dimensions of the actuator pin against the interior dimensions of the valve and the distance that the pin travels are critical in determining the actual amount of fluid dosed at each stroke. The configuration of the pin can also be varied according to the amount of fluid to be dosed.

Variation of dosing can be achieved by varying a number of features of the device, such as:
a) Varying the actuator pin dimensions.
b) Varying the frequency of dose firing.
c) Varying the duration of opening.
d) Varying the outlet dimensions.
e) Varying the viscosity of the formulation.
f) Varying the spring energy available to the progesterone reservoir.
g) Varying the quantity of fill in the progesterone reservoir.
h) Varying the strength of the magnetic field surrounding the rare earth magnet.
i) Varying the strength of the rare earth magnet.

In the illustrated embodiment the progesterone outlet 20 is located directly to the side of the valve arrangement and transfers progesterone formulation from the valve to the outside of the device at the same position on the body as the valve is located. The outlet features a reducing-diameter outlet channel 14, which in turn provides sufficient resistance to the progesterone fluid so that a positive “squirt of formulation” is achieved. By restricting the end of the outlet tube the flow and quantity of progesterone formulation delivered at any one time is able to be governed more accurately.

The reducing-diameter outlet channel 14 commences at approximately 1 mm (over distance 53), reducing to approximately 0.7 mm (over distance 54) and then further reducing to approximately 0.5 mm (over distance 55) to the outside surface of the front body portion and outlet hole 20. The outlet is stepped to the final diameter of 0.5 mm to provide back pressure on the valve for the purpose of providing a satisfactory squirt as the fluid leaves the outside of the device.

The small-diameter side outlet 20 from the progesterone delivery apparatus also limits the opportunity for animal fluid to travel into the valve via the outlet. Fluid ingress could potentially comprise the integrity of dosing.

The invention also features delivery apparatus for delivering the single-unit doses. A mechanical plunger-type delivery system is employed to allow the contents of the single-unit doses reservoirs/conduits to be expelled some distance and with some force from the device. A separate delivery apparatus is used to effect delivery of the separate unit doses, from separate outlets in the device nose cap 6. However, in some embodiments, one plunger system may be used to expel the contents of more than one conduit. The mechanical delivery systems of the invention also controlled by a complementary control system enabling variation in the delivery regime to be effected as required.

In further embodiments which optionally provide for the passive release of a substance (relying on dissolution of the substance by the naturally occurring fluids in the animal's cavity) a collapsible 8 or single-unit type 22 reservoir or conduit may be adapted. The substance may be in a solid form, or as a series of stackable tablets as further discussed below in relation to the single unit doses. Positive pressure may be applied to the rear of the reservoir or conduit by adapted spring and/or plunger tension to continuously present the substance to an outlet as the surface of the substance dissolves. This embodiment is illustrated in part in FIG. 2 as an enlargement of a unit dose conduit with a spring 52 associated therewith. However, such an arrangement may be in a separate location from the unit dose conduits at the front end of the device. Further, the equivalent of the hinged covers of the unit dose may be used with this embodiment, but may feature an aperture to allow passage of the dissolved active into the cavity.

In the illustrated embodiments of the intra-vaginal delivery device, the single unit dose actives are contained within conduits (nose pots) located in the anterior (nose cone portion 5) of the front portion 3 of the device. However, in other embodiments, the single unit dose pots may be located anywhere in the device, as may be the liquid active reservoirs.

Three nose pots (two containing oestradiol and one for prostaglandin) are incorporated into the delivery device design illustrated. These nose pots are capable of housing solid tablets. The tablets are delivered into the animal via a plunger system 25. Spring tension 56 is applied to the plunger (as shown in FIG. 2). The plunger 25 (as shown in FIG. 8) when fired, fully extends to the front of the device (so that the plunger head 57 is flush with the front of the chassis) so that the plunger does not exit the device after firing. The plunger has a close tolerance to the conduit wall, minimising the amount of animal fluid that may ingress while the device is in situ.

The plunger 25 is held under tension in its pre-delivery position. The plunger mechanism is preferably released via heat applied to a thinner portion 58 of the plunger stem. Heat is applied by resistors 60 powered by a battery 31 (shown in FIG. 7). The heat effectively melts the thinner nylon stem portion 58 of the plunger and releases it. A spring 56 associated with the plunger propels the plunger along the conduit 22 and applies pressure to the unit dose causing it to be expelled under force from the unit dose pots, (as partially illustrated in FIG. 2).

A plunger tail 59 of approximately 0.8 mm is located at the rear end of the plunger and allows assembly workers to grasp the plunger without damaging the plunger.

As mentioned previously, each individual unit dose chamber is preferably closed in a substantially fluid tight manner via a cap 23. The cap is preferably resiliently hinged at 24 to the body of the nose of the device, at at least one location around its perimeter. Santoprene™ is the preferred material for the cap. When the plunger system is fired the hinge portion 24 on one side of each individual cap retains the cap and prevents inadvertent loss. The cap provides a snug push-fit to each of the unit-dose chambers and provides an effective seal against for the nose pot reservoirs 22.

The control means effecting control of delivery is an electrical control system. The control means not only controls delivery of the various active ingredients/formulations from the delivery device by instructing the delivery apparatus to release the formulations into the animal according to a predetermined specification, but triggers operation of the device per se, and is able to be reprogrammed to effect recalibration of the delivery apparatus to further regulate delivery of the formulations.

The control of delivery is gained through specialist dose control software that is programmed into a microchip and provides the instructions for the device and determines when the delivery activities will occur. The microchip is energised by a miniature power cell 31 (which provides energy so that the activities can occur) and controls operation through a printed circuit board 32 and its components (not shown in detail) and an electromagnetic coil wound around the chassis at 52 and the on/off switch 61 (which activates the device and determines the time of the delivery process commencing).

The on/off system control means is always related to the printed circuit board and the activation of the printed circuit board. That is, before the device will operate the circuit board must be activated, either from a shutdown or a standby mode.

In the illustrated embodiment (in FIGS. 1,2,5 and 7) an integral on/off switch is located on the top side of the circuit board. The device may be activated manually at the time of insertion of the device by depressing the switch manually for approximately two seconds. Depression of the switch by the user, is effected through a soft, plastic membrane on the outside of the body. When the on/off switch is depressed for the required time, an LED will flash, confirming operation. The software used in the current version allows the operator to turn the device off by, again, depressing the on/off switch for a pre-determined time period.

The integrated circuit board contributes to the operation of the control sequence. The printed circuit board is assembled as an integral unit and clips directly into a strengthened bulkhead at the front end of the chassis.

The circuit board also receives portions of the delivery apparatus for the unit dose pots. Positive alignment of a thinner portion 58 of the plunger straps of the unit dose pot delivery apparatus is effected over the resistors 60 on the front edge of the circuit board 32. As the resistors heat up, they cause the plunger straps to melt and effect operation of the delivery apparatus to deliver the appropriate unit dose into the animal.

The circuit board 32 also includes welded tabs from either end of the power cell 31 that are soldered to the circuit board.

The control means includes a microchip that has software specifically developed to regulate the time and operation and triggering of the delivery apparatus for specific dosing regimes. The microchip is capable of being reprogrammed as required to reflect recalibration of the dose regime thereby actually altering the size of the dose as well as the timing and duration of the dose depending upon the environment the device is in. An appropriate microprocessor for use with the present invention is a four bit microprocessor or an 8 bit single chip microprocessor such as a pic 16cb54 or Z8 or Motorola 6805™.

The control means is also powered by an energy source. In preferred embodiments, the control mechanism is powered by an independent power cell 31 that provides energy to the circuit board. The power cell 31 preferably used is a 3v specialist lithium power cell, which features welded tabs on either end that are in turn soldered to the circuit board.

The control means provides active control over the introduction of the substances. Active operation of the pressure devices means the rate and volume of a substance being delivered may be controlled, so that the precise concentration of the substance can be delivered at the precise time, independent of the environment surrounding the substance delivery device, or delivery apparatus.

The means by which the microprocessor can control the pumps/pressure devices of the delivery apparatus can vary. The pump may be powered by an energy source (perhaps a battery, an external source such as the animal's natural body temperature, a magnetic field, a spring or the like) and the action of the microprocessor may be merely to connect or disconnect the pumps from the energy source. Alternatively, the microprocessor may control the valve or valves which permit or prevent the flow of substance from the delivery apparatus, and substance delivery device.

Accordingly, the delivery regime can follow any sequence depending on the dose control software programmed into the microchip. With the preferred delivery device, dose control is exerted over the time at which a unit dose is delivered (for oestradiol and prostaglandin unit doses), and over the timing and dose volume of a continuous series of pulsatile doses (for progesterone doses).

The microprocessor is programmed to control the release of varying doses of the different hormones into the animal at predetermined times, thus allowing for accurate determination of when oestrus occurs. For example, the release mechanism for firing the front single-unit doses is located at the front of the circuit board. Therefore, the control mechanism becomes part of the delivery system in this instance. At an appropriate time, the control mechanism activates the power (electricity) to the relevant point on the circuit board that, in turn, releases the single-unit dose. With regard to the progesterone delivery, similarly, the control mechanism energises the progesterone delivery system by sending an electrical current to the pump/valve system.

It should also be understood that the term “comprise” where used herein is not to be considered to be used in a limiting sense. Accordingly, ‘comprise’ does not represent nor define an exclusive set of items, but includes the possibility of other components and items being added to the list.

This specification is also based on the understanding of the inventor regarding the prior art. The prior art description should not be regarded as being an authoritative disclosure of the true state of the prior art but rather as referring to considerations in and brought to the mind and attention of the inventor when developing this invention.

Aspects of the present invention have been described by way of example only and it should be appreciated that modifications and additions may be made thereto without departing from the scope thereof, as defined in the appended claims.

1. Substance delivery device capable of insertion into a body cavity of an animal, said delivery device including
a body, the body capable of housing a delivery apparatus capable of actively being controlled to autonomously deliver at least one substance into the animal's body cavity,
said delivery apparatus including dedicated pressure systems to deliver the substance(s) from independent reservoirs via associated outlet(s), said substance(s) ranging in form from substantially fluid to substantially solid,
the device also including programmable control means capable of initiating and regulating delivery of the substance(s) in accordance with a preferred delivery regime,
the body further including a tail portion adapted to receive retention apparatus external to the animal and capable of effecting retention of the substance delivery device within the body cavity of an animal.

2. Substance delivery device as claimed in claim 1 wherein the body is a substantially hollow receptacle including at least two separate but interconnectable portions, said body being adapted to enclose in a substantially fluid tight manner the at least one independent reservoir and an internal chassis of the device with which the delivery apparatus and control means are associated and said body further adapted to receive the tail portion of the device and to include provision for the outlet(s) of the at least one reservoirs.

3. Substance delivery device as claimed in claim 1 wherein the delivery apparatus includes at least one conduit/reservoir capable of containing the substance, at least one pressure device capable of applying pressure to the conduit/reservoir, and valve means,
wherein the delivery apparatus applies pressure by the pressure device and activation of the valve means causing the substance within the conduit/reservoir to move along the conduit/reservoir to the outlet and in to the cavity and wherein the pressure device includes at least one of a pump, a spring, a plunger.

4. Substance delivery device as claimed in claim 1 wherein the independent reservoirs contain substances in substantially fluid or substantially solid form in a fluid tight manner, said reservoirs including any one or a combination of substantially flexible, collapsible, and rigid portions and adapted to release the substance in whole or part in accordance with activation of the delivery apparatus.

5. Substance delivery device as claimed in claim 1 wherein the outlet(s) are adapted to enable delivery of the substances in either or both substantially fluid and substantially solid form during operation of the delivery apparatus, said outlets including at least one of sealing means and restricting means to effect control of release of the substances as required.

6. Substance delivery device as claimed in claim 1 wherein the programmable control means for initiating and regulating delivery of the substance(s) is electric in operation and includes:
a power source,
a microprocessor able to run software for determining and controlling the delivery of a dose by the substance delivery device according to a pre-determined delivery regime,
a printed circuit board including components for effecting operation of either or both resistors and an electromagnetic coil in response to the software being run by the microprocessor, their operation resulting in autonomous delivery of at least one substance from at least one reservoir in accordance with the aforesaid predetermined delivery regime, and
a switch to activate the substance delivery device and/or initiate timing of the delivery of the substance(s) in accordance with the aforesaid predetermined delivery regime.

7. Substance delivery device as claimed in claim 1 wherein the preferred delivery regime includes delivery of substances in any one of:
regulated doses,
at specific concentrations, and
at specific intervals over a predetermined period of time to effect a desired physiological response in an animal for which it is intended to be used.

8. Substance delivery device as claimed in claim 7 in which a desired physiological response includes at least one of:
synchronizing oestrus for effecting controlled breeding programs,
the control of parasites,
affecting growth, and
altering nutritional status.

9. Substance delivery device of claim 7 in which the delivered substance includes at least one of:
a. an oestrogen or oestrogen derivative,
b. prostaglandin or a prostaglandin derivative,
c. progesterone or progesterone derivative,
d. a hormone other than an oestrogen, prostaglandin, or progesterone or derivatives thereof,
e. a vitamin, mineral, or nutritional supplement,
f. a medicine,
g. a parasiticide or insecticide,
h. a prophylactic agent; and
i. a carrier compound, preparation, or solution.

10. Substance delivery device as claimed in claim 1 wherein the range of form of the substances includes liquids of varying viscosities, pastes, gels, powder, granules, capsules/tablets, gases.

11. Substance delivery device as claimed in claim 1 wherein the said tail portion includes a substantially elongate shaft portion and an angled distal portion, said shaft portion being adapted to engage the body of the device and said angled distal portion adapted to receive the retention means;
said tail portion being configured to facilitate insertion and withdrawal of the device in to and from the body cavity of said animal.

12. Substance delivery device as claimed in claim 1 wherein the retention apparatus external to the animal for effecting retention of the substance delivery device within the animal includes, at one distal end, a portion adapted to engage the tail portion of the substance delivery device and, at the opposite end, a portion adapted to engage an exterior surface of the animal.

13. Substance delivery device as claimed in claim 1 wherein the body cavity of an animal includes any one of the ear, the buccal cavity, the vagina, the rumen, and the rectum.

14. Substance delivery device as claimed in claim 1 wherein the operation of delivery apparatus and programmable control means together cause controlled delivery of the at least one substance from the device to the animal in accordance with the preferred delivery regime, in predetermined quantities, for predetermined dose duration, at predetermined times to achieve desired concentrations of the substance in the animal to effect a desired outcome.

15. Substance delivery device as claimed in claim 14 wherein the at least one substance is delivered autonomously in situ to the animal in either or both substantially solid form and substantially fluid form.

16. Substance delivery device as claimed in claim 1 wherein the substance delivery device insertable into an animal's body cavity is adapted to be used at least as an intra-vaginal, intra-rumenal, or intra rectal substance delivery device.

17. Substance delivery device as claimed in claim 16 wherein where the substance delivery device is an intra-vaginal delivery device the substance delivery device is adapted to deliver formulated hormones in regulated doses at specific times in accordance with a predetermined delivery regime into the anterior vagina of an animal to synchronize oestrus for purposes including effecting controlled breeding programs.

18. Substance delivery device as claimed in claim 17 wherein synchronizing oestrous to effect controlled breeding enables fixed time blanket insemination in either cycling or non-cycling animals, including cows.

19. Substance delivery device as claimed in claim 17 wherein the formulated hormones delivered in regulated doses at specific times in accordance with a predetermined delivery regime include four separate hormonal treatments formulated to effect the desired outcome when delivered in precisely the required dose and at the exact time during a specific treatment period.

20. Substance delivery device as claimed in claim 19 wherein the treatment period includes delivery of the hormone formulation treatments over either nine days or eleven days.

21. Substance delivery device as claimed in claim 17 wherein the delivery device is adaptable to deliver a range of formulations into other areas of the animal's body to control or synchronize other body functions or cycles.

22. Substance delivery device as claimed in claim 1 wherein the retention apparatus is adapted to maintain the substance delivery device in situ within the period over which the device is required to deliver the substance(s) from the substance delivery device.

23. Substance delivery device as claimed in claims 16 wherein where the substance delivery device is an intra-vaginal delivery device the retention apparatus is adapted to accommodate the peristaltic nature of the smooth muscle of the anterior vagina and effect retention of the device during the period the device is inserted in the animal.

24. Substance delivery device as claimed in claim 2 wherein the at least two separate, but inter-connectable portions of the body include an anterior front portion at the leading end of the device and a posterior rear portion at the trailing end of the device, when the device is inserted into the animal.

25. Substance delivery device as claimed in claim 24 wherein the front portion of the body is substantially elongate and cylindrical in shape, having a substantially uniform cross-sectional profile, said configuration being dictated by the internal components of the device housed therein including the number of substance formulations and form of the substances to be delivered, the number and structural configuration of reservoirs required to house the formulations, the delivery apparatus (both type and quantity) required to deliver the formulations, the number and location of the outlets for the substances, the control means for effecting the delivery regime of the formulations.

26. Substance delivery device as claimed in claim 24 wherein the rear portion is a substantially ovoid cylinder and being of substantially wider diameter and substantially shorter than the front portion said configuration being dictated by the internal components of the device housed therein including the number of substance formulations and form of the substances to be delivered, the number and structural configuration of reservoirs required to house the formulations, the delivery apparatus (both type and quantity) required to deliver the formulations, the number and location of the outlets for the substances, the control means for effecting the delivery regime of the formulations.

27. Substance delivery device as claimed in claim 24 wherein the configuration of the body portions is determined by one or more of the cavity structure, the physiology of the animal into which the device is being inserted, the process for which the device is being used and the length of time the device is to be retained in the animal.

28. Substance delivery device as claimed in claim 24 wherein the front body portion includes a substantially thinner, indented portion coinciding with an activation switch of the control means to allow an operator to activate the control means of device externally by depressing the switch through the thinner body wall before the device is inserted into the animal.

29. Substance delivery device as claimed in claim 28 wherein the substantially thinner, indented portion of the front body portion also improves operator visibility of the internal components of the device, including either or both a light emitting diode (LED) display and an indicator light, to indicate activation of the control means.

30. Substance delivery device as claimed in claim 24 wherein the front portion of the body is further adapted to include orifices/apertures that form the outlet(s) associated with reservoirs dedicated to containing separate substances and from which the substances in the reservoir(s) are delivered, even though the reservoir for the particular substance may not necessarily be encased by the front body portion.

31. Substance delivery device as claimed in claim 30 wherein a reservoir also operates as a conduit.

32. Substance delivery device as claimed in claim 30 wherein the front portion is further adapted to receive a nose cap portion substantially at the leading distal end, said nose cap portion being adapted to include a plurality of outlet orifices.

33. Substance delivery device as claimed in claim 32 wherein the nose cap portion includes hinged, releasable sealing means to enable delivery of the substances from the reservoirs when required in accordance with the delivery regime.

34. Substance delivery device as claimed in claim 33 wherein the sealing means when sealed provides a substantially fluid tight seal to each of the substance reservoirs.

35. Substance delivery device as claimed in claim 33 wherein release of the releasable sealing means occurs in conjunction with a controlled operation of the delivery pressure devices associated with each reservoir allowing the contents of the substance reservoirs to be expelled from the reservoirs/conduits and delivered into the animal.

36. Substance delivery device as claimed in claim 2 wherein the rear body portion encloses at least one substance reservoir for housing one or more substances in substantially fluid (including pastes or gels), substantially solid (including powder or capsules/tablets) or substantially gaseous form.

37. Substance delivery device as claimed in claim 36 wherein where the at least one substance reservoir(s) when housing a substance in substantially fluid form is adapted to be collapsible.

38. Substance delivery device as claimed in claim 37 wherein where the reservoir is collapsible a breather hole is included in a rear portion of the body to enable air pressure within the device to equilibrate as the collapsible reservoir is emptied during delivery of the substance.

39. Substance delivery device as claimed in claim 36 wherein the rear portion is further adapted to receive a distal end of a shaft of the tail portion.

40. Substance delivery device as claimed in claim 39 wherein the distal end of the shaft of the tail portion is received into a recess in the rear body portion during assembly of the device and provides a secure anchor point for attachment of the retention apparatus to the device.

41. Substance delivery device as claimed in claim 40 wherein the rear body portion is adapted to be strengthened via a series of ridges to provide more rigidity to the rear portion, for supporting either or both reservoir(s) and the tail portion.

42. Substance delivery device as claimed in claim 24 wherein the front and rear body portions are interconnectable via complementary configured interlocking recesses and protrusions molded into the body portions.

43. Substance delivery device as claimed in claim 24 wherein the body portions are adapted to improve the ease with which the substance delivery device is able to be inserted into and withdrawn from a passage or body cavity of an animal, and without causing damage and/or discomfort to the animal via a substantially smooth external finish in conjunction with a substantially cylindrical shape.

44. Substance delivery device as claimed in claim 2 wherein the chassis of the device serves as the internal skeleton of the device to which other internal components are attached, including at least three reservoirs located at variable locations within the body of the device, electronic control means including a printed circuit board and energy source and valve and delivery pressure systems for the reservoirs.

45. Substance delivery device as claimed in claim 44 wherein the body includes at least two portions, the chassis slots into the front portion of the body via a locating lug and is retained in place via a spring/resilient system which forces the chassis against the front body section, and via sealing means including O-ring(s).

46. Substance delivery device as claimed in claim 45 wherein the O-rings are adapted to cause a fluid-tight seal to prevent moisture from the animal's cavity accessing the internal working mechanism including the electronics of the device, particularly where the body includes venting holes to allow atmospheric pressure to equilibrate delivery of substances from collapsible reservoirs and in doing so allow animal fluids into the device.

47. Substance delivery device as claimed in claim 11 wherein the substantially internal shaft portion of the tail portion is positioned in situ internally of the cavity and the angled distal portion at an outer distal end of the shaft is positioned adjacent the entrance of the cavity and includes the engaging means adapted to receive a portion of the retention apparatus to be located externally of the cavity.

48. Substance delivery device as claimed in claim 47 wherein the external distal portion is angled at a right angle to the shaft.

49. Substance delivery device as claimed in claim 47 wherein the tail portion for use with the delivery device to be inserted into the vagina of a cow includes a substantially elongate shaft that extends from the rear of the body of the device by a distance of approximately 126 mm, is substantially circular in a cross-section and is approximately 14 mm in diameter along its length and wherein the external distal portion is angled at right-angles to the shaft and extends for approximately 12 mm to barbed engaging means capable of engaging with a portion of the retention apparatus.

50. Substance delivery device as claimed in claim 49 wherein the right angled distal portion located at the outer distal end of the tail is further adapted to determine the depth of insertion of the device as the right-angled distal portion is located adjacent the vulval lips of the animal.

51. Substance delivery device as claimed in claim 49 wherein the substantially elongate shaft is adapted to limit the delivery device being dislodged or expelled from the anterior vagina of the animal, to minimize internal pressure against the vaginal wall while the device is in situ, ensures that the device remains in the correct orientation and location while in situ, ensures minimal trauma to the animal during insertion and removal and avoids the need for an applicator to insert the device into the animal.

52. Substance delivery device as claimed in claim 51 wherein the substantially elongate shaft is configured to include substantially straight, spiraled, curved, helical and/or include undulating portions along all or part of its length, and/or be extendible telescopically, to enable it to be adapted to complement a range of sizes of body cavities and/or the physiology associated with different body cavities of different animals into which the delivery device may be inserted.

53. Substance delivery device as claimed in claim 49 wherein the tail of the device contributes to retention of the device within the animal's cavity particularly where the cavity is the vagina, using an internal connecting portion between the retention apparatus and the device.

54. Substance delivery device as claimed in claim 11 wherein the tail serves as the applicator for the device by enabling an operator inserting the device to hold the tail at the time of insertion of the device and to use it to guide and deliver the device to a correct location within the animal.

55. Substance delivery device as claimed in claim 11 wherein the tail serves to facilitate withdrawal of the device when the functional utility of the device has been exhausted by an operator pulling on the tail portion which when further assisted by pressure exerted by the walls of the passage or the opening to the body cavity enables the device to be withdrawn with minimum discomfort to the animal.

56. A method of inserting into and withdrawing from a body cavity of an animal a substance delivery device, using the tail of the animal.

57. Substance delivery device as claimed in claim 11 wherein the distal end of the shaft of the tail portion is attached to the rear portion of the body of the device by any one of:
a) being rigidly fixed into the rear portion of the body of the device;
b) being molded onto the body of a device; and
c) being press-fitted into an aperture in the rear portion of the body.

58. Substance delivery device as claimed in claim 57 wherein distal end of the shaft of the tail portion is attached to the rear portion of the body of the device such that when also connected to the retention apparatus the orientation of the tail is predetermined and ensures the device has the same orientation within the cavity in all instances.

59. Substance delivery device as claimed in claim 57 wherein the tail is adapted to be located in the chassis of the device.

60. Substance delivery device as claimed in claim 57 wherein when the distal end of the shaft of the tail portion is press-fitted into the aperture in the rear portion of the body the distal end is secured in place via protrusions, including barbs, which positively engage the body.

61. Substance delivery device as claimed in claim 58 wherein the tail portion is adapted to be detachable from the body of the device and/or be re-usable.

62. Substance delivery device as claimed in claim 49 wherein the tail includes an exterior surface adapted to be smooth so as minimize abrasion or trauma with the target animal, and to ensure that any animal excrement, mucous or body fluid does not bond or bind to the tail once inserted and while in situ, whilst also being dependent on the internal conditions of the body cavity into which the device is inserted, and the corresponding shaft strength required.

63. Substance delivery device as claimed in claim 11 wherein to facilitate use of the tail portion for insertion or withdrawal of the substance delivery device into the vaginal body cavity of an animal the tail portion is substantially rigid to enable the shaft of the tail portion to withstand stress without the shape of the shaft becoming distorted.

64. Substance delivery device as claimed in claim 63 wherein to provide a substantially rigid tail portion, the tail portion is manufactured from thermoplastics materials including a glass component of between 5% to 40%.

65. Substance delivery device as claimed in claim 64 wherein the tail portion includes a glass content of less than 25% for use when greater flexibility of the tail portion is required in the insertion of the device, or for use when the retention of the device in the animal for the length of time required to effect delivery of the substances into the animal may be less crucial.

66. Substance delivery device as claimed in claim 64 wherein the tail portion includes a glass content of greater than 33% where a more rigid tail portion is required and it is not crucial for a smooth surface finish to the exterior molding of the tail portion.

67. Substance delivery device as claimed in claim 64 wherein the tail portion is used in conjunction with an intravaginal delivery device, the preferred glass to thermoplastics ratio is 33% glass to 66% nylon to provide a rigid, tail portion with minimal flexibility.

68. A method for retaining a substance delivery device within a body cavity of an animal into which the substance delivery device is inserted, via use of retention apparatus means.

69. Substance delivery device as claimed in claim 12 wherein an external point of attachment of the retention apparatus on the animal is dependent on the animal's behavioral characteristics.

70. Substance delivery device as claimed in claim 69 wherein an external point of attachment of the retention apparatus on naturally passive animals including cows is located on the animal's back.

71. Substance delivery device as claimed in claim 69 wherein an external point of attachment of the retention apparatus on naturally inquisitive animals, including pigs, is in the vicinity of the vulval area, relying on the animal's reluctance to allow other animals to touch that area to minimize the likelihood of another animal pulling off, or chewing the retention apparatus.

72. Substance delivery device as claimed in claim 12 wherein the retention apparatus includes a patch and strap arrangement.

73. Substance delivery device as claimed in claim 72 wherein the strap is substantially elongate and resilient and adapted to attach at one distal end to the angled portion of the tail rod and including the patch at the outer free distal end of the strap.

74. Substance delivery device of claims 72 and claim claim 72 in which the patch for an intra vaginal device is attached to the back of the animal and the strap is under sufficient tension at the time of insertion of the device into the animal's vagina to maintain the device in the animal but without the angled distal portion of the tail portion rubbing against and causing trauma to the animal's vulval commissure.

75. Substance delivery device as claimed in claim 74 wherein the patch when adapted to be attached to the back of the cow is approximately 55 mm-60 mm in diameter.

76. Substance delivery device as claimed in claim 75 wherein patch is attached to the animal's back via adhesive applied to either or both the patch and the surface of the animal.

77. Substance delivery device as claimed in claim 76 wherein patch includes a roughened surface to accentuate bond between the strap and the cow when the adhesive is applied and the patch is glued to the animal.

78. Substance delivery device as claimed in claim 74 wherein the strap tension, the tail dimensions and attachment of the patch to the animal's back minimizes occurrence of expulsion of the device from the animal via muscular peristalsis or accidental removal from the vagina.

79. Substance delivery device as claimed in claim 71 wherein the retention apparatus includes any one of a cap, diaphragm or similar means that applies suction or adhesion around the vulval region and retains the device in situ.

80. Substance delivery device as claimed in claim 53 wherein the retention apparatus is integral with the tail.

81. Substance delivery device as claimed in claim 9 wherein the hormones delivered in regulated doses at specific times in accordance with a predetermined delivery regime include two formulations of estradiol, one of prostaglandin and a progesterone formulation.

82. Substance delivery device as claimed in claim 81 wherein the formulations of estradiol and prostaglandin are delivered from the delivery device via a plurality of delivery apparatus adapted to deliver required single-unit doses at a specific time.

83. Substance delivery device as claimed in claim 81 wherein the formulation of progesterone is delivered from the delivery device via at least one delivery apparatus adapted to deliver a continuous dosing to replicate the naturally occurring situation in the animal.

84. Substance delivery device as claimed in claim 83 wherein the delivery apparatus used to provide continuous dosing of a progesterone formulation includes a collapsible reservoir.

85. Substance delivery device of claim 84 in which the continuous dosing of a progesterone formulation from a collapsible reservoir is achieved by either or both:
active delivery of specific doses from the delivery apparatus at controlled predetermined time intervals, and
passive delivery through controlled dissolution in accordance with a predetermined delivery regime.

86. Substance delivery device as claimed in claim 85 wherein the progesterone available in at least one of a substantially liquid, powdered, or tablet form.

87. Substance delivery device as claimed in claim 86 wherein where the progesterone is adapted for delivery in to the animal's body cavity via one or more of:
being dissolved in a carrier solution in readiness for delivery; or
the carrier solution is stored separately from the progesterone until prior to release of the formulation into the animal, when the carrier solution is introduced to the progesterone with the dissolved final formulation released into the animal; or
the carrier solution is stored separately from the progesterone until release of the formulation into the animal, when the carrier solution is introduced to the progesterone with mixing occurring at the time of release; or where the progesterone is in powdered or tablet form; or
dissolution in the animal's body fluids.

88. Substance delivery device as claimed in claim 87 wherein where progesterone is used in the formulation the carrier solution includes a solvent comprising either or both water and an alcohol to make soluable the progesterone, to facilitate transfer of the hormone across membranes, particularly in substantially aqueous environments of a body cavity, such as the vagina.

89. Substance delivery device as claimed in claim 9 wherein the carrier compound, preparation, solution is included to facilitate the transfer of insoluble, or partially soluble substances across water-soluble membranes and includes chemical compounds capable of forming inclusion complexes.

90. Substance delivery device as claimed in claim 89 wherein at least one chemical compound capable of forming inclusion complexes for transferring insoluble, or partially soluble substances across water-soluble membranes includes cyclodextrin.

91. Substance delivery device as claimed in claim 86 wherein the progesterone is delivered in a substantially fluid form activation of the pressure device of the delivery apparatus effects delivery of the progesterone as a continuous series of pulsatile doses from the reservoir in accordance with the predetermined delivery regime.

92. Substance delivery device as claimed in claim 3 wherein the pressure applied to cause movement of the substance(s) being delivered from the reservoir to the outlet of the substance delivery device is an expulsive force.

93. A method for delivering a substance to an outlet of a substance delivery device including at least one conduit capable of containing the substance, at least one pressure device capable of applying pressure to the conduit/reservoir and valve means, wherein the steps of applying pressure to the conduit/reservoir and valve means, causing the substance within the conduit to move along the conduit to the outlet.

94. Substance delivery device as claimed in claim 86 wherein the progesterone is delivered in a substantially solid form activation of the pressure device of the delivery apparatus effects delivery of the progesterone within the reservoir to an outlet of the substance delivery device where the surface of the progesterone is subjected to passive dissolution by fluids in the cavity.

95. Substance delivery device as claimed in claim 3 wherein tension pressure is applied to either or both the reservoir and the substance to effect movement of the substance(s) being delivered from the reservoir to the outlet of the substance delivery device.

96. A method for delivering a substance to an outlet of a substance delivery device including at least one conduit capable of containing the substance, at least one pressure device capable of applying pressure to either or both the conduit/reservoir and the substance therein, wherein the steps of applying pressure to effect movement of the substance within the conduit to the outlet.

97. Substance delivery device as claimed in claim 3 wherein the pressure device is activated by a power source, including a battery.

98. Substance delivery device as claimed in claim 84 wherein the collapsible reservoir containing the progesterone is maintained under positive pressure by means of a spring force applied to the posterior end of the reservoir such that the substance is always presented to the inlet of a pump to which the bellows reservoir is attached, regardless of the attitude of the device, used to effect continuous programmed series of pulsatile doses of the progesterone.

99. Substance delivery device as claimed in claim 84 wherein the collapsible reservoir containing the progesterone is maintained under positive pressure by means of a spring force applied to the posterior end of the reservoir such that the substance is always presented to an outlet of the reservoir, regardless of the attitude of the device, when used to effect continuous passive doses.

100. Substance delivery device as claimed in claim 98 wherein to provide delivery of the progesterone solution as a continuous programmed series of pulsatile doses according to command, pressure is applied to the posterior end of the collapsible reservoir by a seal and spring such that a valve is opened for a specific period of time to allow some of the contents of the reservoir to fill a small delivery cavity and whereby pressure from repeated operation of the valve effectively drives the fluid to the outlet.

101. Substance delivery device as claimed in claim 100 wherein the pump is operated by means of a magnetic core that is forced to stroke when a electromagnetic coil, within which the magnetic core is housed, is energized, such that the core activates a release mechanism allowing a predetermined quantity of solution to enter the pump with the positive pressure in the reservoir ensuring the progesterone solution enters the pump and is expelled from the outlet under pressure.

102. Substance delivery device as claimed in claim 84 wherein the reservoir is adapted to collapse via the inclusion of annular rings that enable the reservoir to concertina when even spring pressure is applied at the posterior end of the reservoir thereby effecting movement of the substance along the reservoir.

103. Substance delivery device as claimed in claim 102 wherein the posterior end of the reservoir is further adapted to effect even collapsing via reinforcing means thereby minimizing racking, implosion from the posterior end of the reservoir, or uneven dosing or output from the device.

104. Substance delivery device as claimed in claim 91 wherein the reservoir contain progesterone in substantially fluid form the quantity of fluid required to fill the reservoir optimally ranges between 12 ml and 42 ml with the reservoir being varyingly configured to accommodate the fluid.

105. Substance delivery device as claimed in claim 104 wherein the fluid contained in the reservoir is directed towards the anterior end of the reservoir when the reservoir collapses, and towards valve means also located at the anterior end of the reservoir.

106. Substance delivery device as claimed in claim 105 wherein the bellows is adapted to be connected directly to the valve means.

107. Substance delivery device as claimed in claim 106 wherein the valve means includes a central aperture, a seal positioned to seal the aperture and a valve ring to lock the seal in a fixed position.

108. Substance delivery device as claimed in claim 107 wherein the collapsible reservoir is substantially tapered adjacent the valve means to effect maximum delivery of the progesterone in fluid form to the valve means and to minimize residual progesterone in the reservoir at the end of the dosing regime.

109. Substance delivery device as claimed in claim 108 wherein the seal of the valve means is sealed via a spring applied against the rear of the reservoir effecting pressure from the progesterone fluid against the seal thus keeping the valve closed at all times while the valve is not being operated, yet during operation of the valve means the seal provides the energy to push the progesterone fluid to the outlet.

110. Substance delivery device as claimed in claim 109 wherein the valve means is a metering valve system operating on a reversed magnetic polarity principle, including either a moving or a fixed armature capable of being magnetized, or a rare earth magnet and an actuator pin which opens the valve means, a coil member capable of being magnetized, tension apparatus, and a chamber capable of receiving a substance from a conduit/reservoir.

111. Substance delivery device as claimed in claim 110 wherein the valve means comprises a mild steel bobbin with an attached seal, which moves on a horizontal plane when a surrounding coil member capable of being activated, is energized creating a solenoidal effect resulting in the moving armature sealing against the stationary armature, thus reversing the polarity of a rare earth magnet providing energy to the valve means and momentarily opening the seal between the valve and a bulkhead, allowing fluid to travel through a narrow conduit to an outlet of the device, with deactivation of the coil member enabling the moving armature to move away from the fixed armature.

112. Substance delivery device as claimed in claim 111 wherein the coil member is a copper coil located substantially adjacent to the chassis to provide the energy to the magnet upon activation.

113. Substance delivery device as claimed in claim 111 wherein the metering valve system is adapted to create a chamber within the conduit, such that movement of the moving armature towards and away from the fixed armature enables some of the substance which has passed into this chamber to move along the conduit to the outlet, thereby operating as a pump.

114. Substance delivery device as claimed in claim 110 wherein the valve system includes a rare earth magnet and an actuator pin which opens the valve at the time of activation of the coil member the polarity of the rare earth magnet is reversed causing the magnet to strike the actuator pin which in turn opens the seal on the valve arrangement.

115. Substance delivery device as claimed in claim 112 wherein activation of the coil member is controlled by the operation of a software program for a controlled duration to enable fluid to travel past the actuator pin into the magnet chamber and towards the outlet.

116. Substance delivery device as claimed in claim 113 wherein the progesterone formulation is in contact with the magnet during delivery.

117. Substance delivery device as claimed in claim 114 wherein the actuator pin is configured to complement the interior dimensions of the valve means to ensure that the seal is opened for a predetermined period at each stroke of the pin.

118. Substance delivery device as claimed in claim 117 wherein the dimensions of the actuator pin against the interior dimensions of the valve means and the distance the pin travels are determine the actual amount of fluid dosed at each stroke.

119. Substance delivery device as claimed in claim 118 wherein the dimensions of the actuator pin are capable of being variably selected according to the amount of fluid to be dosed.

120. Substance delivery device as claimed in claim 119 wherein variation to the amount of fluid to be dosed is achievable by varying any one or more of:
the actuator pin dimensions,
the frequency of dose firing,
the duration of opening,
the outlet dimensions,
the viscosity of the formulation,
the spring energy available to the progesterone reservoir,
the quantity of fill in the progesterone reservoir,
the strength of the magnetic field surrounding the rare earth magnet, and
the strength of the rare earth magnet.

121. Substance delivery device as claimed in claim 120 wherein variation to the amount of fluid to be dosed is achievable by varying the dimensions of the actuator pin by any one of:
lengthening the fine point of the pin which strikes the seal thereby opening the seal further to enable more fluid to be dosed at each stroke of the pin, and
shortening the body of the pin such that the magnet has to travel further and a longer lag time in opening of the valve occurs.

122. Substance delivery device as claimed in claim 115 wherein the outlet for progesterone delivery is located substantially adjacent the valve means for ensuring active delivery of the progesterone formulation to the target area within the body cavity.

123. Substance delivery device as claimed in claim 122 wherein the outlet includes a reducing-diameter outlet providing sufficient resistance to the progesterone fluid to effect one or more of:
a positive “squirt of formulation”,
control over the flow and quantity of progesterone formulation delivered at any one time, and
consistency of small-dose outputs on an accurate basis.

124. Substance delivery device as claimed in claim 123 wherein the reducing-diameter outlet is reduced through three reductions from 1 mm, to 0.7 mm, to 0.5 mm on the external surface of the body of the device to provide a back pressure on the valve means to cause active expulsion as the fluid leaves the device.

125. Substance delivery device as claimed in claim 124 wherein the device is an intravaginal device the progesterone formulation is actively expelled from the device to clear the device and any mucous naturally accumulating around the device in the anterior vagina.

126. Substance delivery device as claimed in claim 82 wherein to minimize the effects of the accumulation of mucous and to ensure a formulation is optimally presented in the cavity for transfer across the vaginal mucosa, the delivery apparatus for the single-unit doses are adapted to be a plunger-type delivery system to expel the single unit doses some distance and with some force from the device.

127. Substance delivery device as claimed in claim 126 wherein the single-unit doses of formulation are delivered as any one or more of a tablet, a capsule, or liquid formulation of varying viscosity.

128. Substance delivery device as claimed in claim 127 wherein for controlling oestrus in cattle, a plurality of doses of oestradiol and prostaglandin in individual solid capsules are contained within three single dose conduits located in the anterior of the delivery device with at least one pressure device associated with each conduit.

129. Substance delivery device as claimed in claim 127 wherein for controlling oestrus in cattle, a plurality of doses of progesterone in individual solid capsules, relying on passive dissolution within the animal's vagina, are also optionally contained within single dose conduits with at least one pressure device associated with each conduit.

130. Substance delivery device as claimed in claim 128 wherein the at least one pressure device of the unit doses includes a plunger that operates between a pre-delivery position and a fired position.

131. Substance delivery device as claimed in claim 130 wherein when the plunger is in a pre-delivery position the plunger is held under tension until released.

132. Substance delivery device as claimed in claim 130 wherein when the plunger is in a fired position the plunger fully extends to the front of the device.

133. Substance delivery device as claimed in claim 131 wherein when the plunger is released heat is applied to a portion of the plunger which melts such that a spring associated with the plunger propels the plunger along the conduit which in turn applies pressure to the unit dose causing it to be expelled under force from the unit dose conduit.

134. Substance delivery device as claimed in claim 133 wherein the heat required to melt the plunger is provided by any one of energy sources within the substance delivery device including battery energy, or energy sources in the surrounding environment including kinetic, chemical, or thermal energy.

135. Substance delivery device as claimed in claim 129 wherein the individual unit dose conduit also includes a nose cap to provide fluid seal.

136. Substance delivery device as claimed in claim 135 wherein the nose cap is resiliently hinged to the body of the device in at least one location around its perimeter.

137. Substance delivery device as claimed in claim 136 wherein the hinge of the nose cap retains the nose cap in place and prevents inadvertent loss of the nose cap after the plunger has fired.

138. Substance delivery device as claimed in claim 1 wherein the pressure devices are either capable of causing the flow of substance through the conduit, and/or capable of causing release of the substance from the conduit without back flow.

139. A method of introducing a substance into an animal from a substance delivery device substantially as described above, wherein the step of controlling at least one of the operation of the delivery apparatus to effect active introduction of a substance in to a cavity, the time of delivery of one or more substances, the volume of substance delivered on a dose by dose basis.

140. Substance delivery device as claimed in claim 1 wherein the control means controls delivery of the substances from the reservoirs by triggering activation of the delivery apparatus in accordance with a predetermined regime.

141. Substance delivery device as claimed in claim 140 wherein the control means is reprogrammable to effect at least one of:
recalibration of the triggering of the delivery apparatus,
regulation of delivery via altering the size of the dose, the timing and/or the duration of the dose, and
responding to environmental changes around the substance delivery device in response to feedback from sensors around the substance delivery device.

142. Substance delivery device as claimed in claim 141 wherein the sensors determine when the environment is ideal for the introduction of a substance into the body of the animal thus enabling the microprocessor to control the delivery apparatus to introduce the substance.

143. Substance delivery device as claimed in claim 142 wherein the sensors determine factors in the body fluid surrounding the substance delivery device and/or delivery apparatus, such as temperature, acidity, viscosity or even odor which act as physiological indicators that may be more accurate than a calendar date for determining when certain substances should be introduced into the animal.

144. Substance delivery device as claimed in claim 6 wherein the switch is activated by:
depression of the switch manually at the time of insertion of the device into the cavity for a preprogrammed time, and
activation, control and programming from a remote source by electronic signaling means.

145. Substance delivery device as claimed in claim 144 wherein activation of the switch is confirmed by a flashing LED display.

146. Substance delivery device as claimed in claim 145 wherein electronic signaling means that is external device to the animal for triggering the activation of the switch includes a radio transmitter, an ultrasonic transmitter, or a magnet.

147. Substance delivery device as claimed in claim 141 wherein the microprocessor runs a software program to effect one or more of:
triggering and/or regulating the action of the pumps/pressure devices of the delivery apparatus,
connecting or disconnecting the pumps from the energy source,
controlling operation of the valve means to permit or prevent the flow of substance from the delivery apparatus and/or from the outlets of the substance delivery device, and
effecting supply of power to resistors on the circuit board to effect release of the mechanism for firing the front single-unit doses.

148. Substance delivery device as claimed in claim 147 wherein the microprocessor and associated software effect activation of the release mechanism for the progesterone delivery by the control mechanism energizing the progesterone delivery system by sending an electrical current to the pump, valve, magnet.

149. Substance delivery device as claimed in claim 147 wherein the microprocessor and associated software effects control over the time at which a unit dose is delivered, and over the timing and dose volume of a continuous series of pulsatile doses.

150. Substance delivery device as claimed in claim 151 wherein microprocessor is a four bit microprocessor or an 8 bit single chip microprocessor such as a pic 16c54 or Z8 or Motorola 6805.

151. Substance delivery device as claimed in claim 6 wherein the power source of the control means is a 3 volt specialist lithium power cell.

152. A method of controlling the delivery apparatus of a substance delivery device wherein the step of introducing predetermined amounts of at least one substance at predetermined times into the body of an animal.

153. A method of delivering a substance to a body wherein the step of using a pressure device of the delivery apparatus to apply pressure to the conduit of the delivery apparatus, causing the substance within the conduit to move along the conduit to an outlet and into a body.

154. A method of introducing a substance into an animal wherein the step of controlling the operation of the substance delivery device so that the substance is actively introduced into the animal.

155. A method of introducing a substance into an animal from a substance delivery device, wherein the step of controlling the operation of the delivery apparatus so that the substance is actively introduced into the animal.

156. A method of controlling the delivery apparatus of a substance delivery device, wherein the step of introducing predetermined amounts of substance at predetermined times into the body of an animal.

157. A method of controlling the introduction of a substance into an animal from a substance delivery device, wherein energy sources in the environment surrounding the substance delivery device are utilized.
